SEQUENCE LISTING (58 sequences)

SEQ ID NO: 1 - DNA, 59 bp, synthetic construct
  source 1..59: mol_type = other DNA
attggcggcgtagtacacactattgaatcaaacagccgaccaattgcactaccatcaca

SEQ ID NO: 2 - DNA, 1620 bp, synthetic construct
  source 1..1620: mol_type = other DNA
atggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagca

SEQ ID NO: 3 - DNA, 2421 bp, synthetic construct
  source 1..2421: mol_type = other DNA
gcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagcc

SEQ ID NO: 4 - DNA, 1683 bp, synthetic construct
  source 1..1683: mol_type = other DNA
gcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtggg

SEQ ID NO: 5 - DNA, 1834 bp, synthetic construct
  source 1..1834: mol_type = other DNA
tacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagt

SEQ ID NO: 6 - DNA, 24 bp, synthetic construct
  source 1..24: mol_type = other DNA
atctctacggtggtcctaaatagt

SEQ ID NO: 7 - DNA, 792 bp, synthetic construct
  source 1..792: mol_type = other DNA
atgaatagaggattctttaacatgctcggccgccgccccttcccagcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcgccaaagcaaccaccgaagccgaagaaaccaaaaacacaggagaagaagaagaagcaacctgcaaaacccaaacccggaaagagacagcgtatggcacttaagttggaggccgacagactgttcgacgtcaaaaatgaggacggagatgtcatcgggcacgcactggccatggaaggaaaggtaatgaaaccactccacgtgaaaggaactattgaccaccctgtgctatcaaagctcaaattcaccaagtcgtcagcatacgacatggagttcgcacagttgccggtcaacatgagaagtgaggcgttcacctacaccagtgaacaccctgaagggttctacaactggcaccacggagcggtgcagtatagtggaggcagatttaccatcccccgcggagtaggaggcagaggagacagtggtcgtccgattatggataactcaggccgggttgtcgcgatagtcctcggaggggctgatgagggaacaagaaccgccctttcggtcgtcacctggaatagcaaagggaagacaatcaagacaaccccggaagggacagaagagtgg

SEQ ID NO: 8 - DNA, 54 bp, synthetic construct
  source 1..54: mol_type = other DNA
gccaactttgacctacttaagttggcgggagacgttgagtccaaccctgggccc

SEQ ID NO: 9 - DNA, 192 bp, synthetic construct
  source 1..192: mol_type = other DNA
tctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaaga

SEQ ID NO: 10 - DNA, 1269 bp, synthetic construct
  source 1..1269: mol_type = other DNA
agcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgct

SEQ ID NO: 11 - DNA, 93 bp, synthetic construct
  source 1..93: mol_type = other DNA
gaaacattcaccgagaccatgagttacctatggtcgaacagccagccattcttctgggtccagctgtgtatacccctggccgctgtcatcgtt

SEQ ID NO: 12 - DNA, 1389 bp, synthetic construct
  source 1..1389: mol_type = other DNA
ctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaaga

SEQ ID NO: 13 - DNA, 318 bp, synthetic construct
  source 1..318: mol_type = other DNA
ccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttatttagcggacgccaaaactcaatgtatttctgaggaagcatggtgcataatgccatgcagcgtctgcataactttttattatttcttttattaatcaacaaaattttgtttttaacatttc

SEQ ID NO: 14 - DNA, 167 bp, synthetic construct
  source 1..167: mol_type = other DNA
tcccctgtgagctagactggacagccaatgacgggtaagagagtgacatttctcactaacctaagacaggagggccgtcaaagctactgcctaatccaatgacgggtaatagtgacaagaaatgtatcactccaacctaagacaggcgcagcctccgagggatgtgt

SEQ ID NO: 15 - DNA, 95 bp, synthetic construct
  source 1..95: mol_type = other DNA
tgtttggcccggtggatggctggttagccagagacgggtaagattcctcagggaaggaacaacctaagacaggcacagtcgcagaggggccatca

SEQ ID NO: 16 - DNA, 35 bp, synthetic construct
  source 1..35: mol_type = other DNA
aaaaaaaaaaaaaaaaaaaaaaaaaaaaaaaaaaa

SEQ ID NO: 17 - DNA, 72 bp, synthetic construct
  source 1..72: mol_type = other DNA
gatggccggcatggtcccagcctcctcgctggcgccggctgggcaacaccttcgggtggcgaatgggacttt

SEQ ID NO: 18 - DNA, 72 bp, synthetic construct; identical to SEQ ID NO: 17
  source 1..72: mol_type = other DNA

SEQ ID NO: 19 - DNA, 13970 bp, synthetic construct
  source 1..13970: mol_type = other DNA
atttaggtgacactatagattggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccagcggccgcatcgatcaccggtcgccaccatgaatagaggattctttaacatgctcggccgccgccccttcccggcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcggccgccaactttgacctacttaagttggcgggagacgttgagtccaaccctgggccctctgctgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccgccaactttgacctacttaagttggcgggagacgttgagtccaaccctgggccctctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaagaagcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgctgaaacattcaccgagaccatgagttacctatggtcgaacagccagccattcttctgggtccagctgtgtatacccctggccgctgtcatcgttctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaagatgaagcggcatcgataccggtccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaggcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaggggatctcgagctctagagctcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtatggacatattgtcgttagaacgcggctacaattaatacataaccttatgtatcatacacatacg

SEQ ID NO: 20 - DNA, 20 bp, synthetic construct
  source 1..20: mol_type = other DNA
cacatgaagcagcacgactt

SEQ ID NO: 21 - DNA, 20 bp, synthetic construct
  source 1..20: mol_type = other DNA
tgctcaggtagtggttgtcg

SEQ ID NO: 22 - DNA, 76 bp, synthetic construct
  source 1..76: mol_type = other DNA
tcgtcggcagcgtcagatgtgtataagagacaggatacnnnyrnnnyrnnnyrnnncacatgaagcagcacgactt

SEQ ID NO: 23 - DNA, 77 bp, synthetic construct
  source 1..77: mol_type = other DNA
gtctcgtgggctcggagatgtgtataagagacaggatacnnnyrnnnyrnnnyrnnntgctcaggtagtggttgtcg

SEQ ID NO: 24 - DNA, 19 bp, synthetic construct
  source 1..19: mol_type = other DNA
ctcgcgaagtgtcctccag

SEQ ID NO: 25 - DNA, 20 bp, synthetic construct
  source 1..20: mol_type = other DNA
attgtgcatgacgttgctga

SEQ ID NO: 26 - DNA, 27 bp, synthetic construct
  source 1..27: mol_type = other DNA
tcgctatactaaccgttacgctgtccc

SEQ ID NO: 27 - DNA, 6186 bp, synthetic construct
  source 1..6186: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtatggacatattgtcgttagaacgcggctacaattaatacataaccttatgtatcatacacatacgatttaggtgacactatagattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactgatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcgccaaagcaaccaccgaagccgaagaaaccaaaaacacaggagaagaagaagaagcaacctgcaaaacccaaacccggaaagagacagcgtatggcacttaagttggaggccgacagactgttcgacgtcaaaaatgaggacggagatgtcatcgggcacgcactggccatggaaggaaaggtaatgaaaccactccacgtgaaaggaactattgaccaccctgtgctatcaaagctcaaattcaccaagtcgtcagcatacgacatggagttcgcacagttgccggtcaacatgagaagtgaggcgttcacctacaccagtgaacaccctgaagggttctacaactggcaccacggagcggtgcagtatagtggaggcagatttaccatcccccgcggagtaggaggcagaggagacagtggtcgtccgattatggataactcaggccgggttgtcgcgatagtcctcggaggggctgatgagggaacaagaaccgccctttcggtcgtcacctggaatagcaaagggaagacaatcaagacaaccccggaagggacagaagagtggtctgctgcaccactggtcacggccatgtgcttgcttggaaacgtgagcttcccatgcaatcgcccgcccacatgctacacccgcgaaccatccagagctctcgacatcctcgaagagaacgtgaaccacgaggaattccaagccgccatctcaaaaacttcatggagttggctgtttgcccttttcggcggcgcctcgtcgctattaattataggacttatgatttttgcttgcagcatgatgctgactagcacacgaagatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaaaaaaaaaaaaaggccggcatggtcccagcctcctcgctggcgccggctgggcaacattccgaggggaccgtcccctcggtaatggcgaatgggacgggaattcctcgagtcttgagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaaataaaacgaaaggctcagtcgaaagactgggcctttcgttttatctgttgtttgtcggtgaacgctctctactagagtcacactggctcaccttcgggtgggcctttctgcggggtttaaactctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 28 - DNA, 6820 bp, synthetic construct
  source 1..6820: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtacaattgcatgaagaatctgcttagggttaggcgttttgcgctgcttcgcgatgtacgggccagatagacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccatgaatagaggattctttaacatgctcggccgccgccccttcccagcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcgccaaagcaaccaccgaagccgaagaaaccaaaaacacaggagaagaagaagaagcaacctgcaaaacccaaacccggaaagagacagcgtatggcacttaagttggaggccgacagactgttcgacgtcaaaaatgaggacggagatgtcatcgggcacgcactggccatggaaggaaaggtaatgaaaccactccacgtgaaaggaactattgaccaccctgtgctatcaaagctcaaattcaccaagtcgtcagcatacgacatggagttcgcacagttgccggtcaacatgagaagtgaggcgttcacctacaccagtgaacaccctgaagggttctacaactggcaccacggagcggtgcagtatagtggaggcagatttaccatcccccgcggagtaggaggcagaggagacagtggtcgtccgattatggataactcaggccgggttgtcgcgatagtcctcggaggggctgatgagggaacaagaaccgccctttcggtcgtcacctggaatagcaaagggaagacaatcaagacaaccccggaagggacagaagagtggtctgctgcaccactggtcacggccatgtgcttgcttggaaacgtgagcttcccatgcaatcgcccgcccacatgctacacccgcgaaccatccagagctctcgacatcctcgaagagaacgtgaaccacgaggaattccaagccgccatctcaaaaacttcatggagttggctgtttgcccttttcggcggcgcctcgtcgctattaattataggacttatgatttttgcttgcagcatgatgctgactagcacacgaagatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttatttagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaagatggccggcatggtcccagcctcctcgctggcgccggctgggcaacaccttcgggtggcgaatgggactttggtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 29 - DNA, 12060 bp, synthetic construct
  source 1..12060: mol_type = other DNA
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccagcggccgcatcgatcaccggtcgccaccatgaatagaggattctttaacatgctcggccgccgccccttcccggcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcggccgccaactttgacctacttaagttggcgggagacgttgagtccaaccctgggccctctgctgcaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggccgccaactttgacctacttaagttggcgggagacgttgagtccaaccctgggccctctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaagaagcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgctgaaacattcaccgagaccatgagttacctatggtcgaacagccagccattcttctgggtccagctgtgtatacccctggccgctgtcatcgttctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaagatgaagcggcatcgataccggtccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaggcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaa

SEQ ID NO: 30 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 31 - DNA, 13998 bp, synthetic construct
  source 1..13998: mol_type = other DNA
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccagcggccgcatcgatcaccggtcgccaccatgaatagaggattctttaacatgctcggccgccgccccttcccggcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcggccgccaactttgacctacttaagttggcgggagacgttgagtccaaccctgggccctctgctgcaatggtccaaactagttcgaagatctccggagctcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtgcctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagagcgctggatggttgatacaggtatcgaagtacctgagcaaggggagttcgaccttaacgaaatcatggattctgaatacgtatttgccaaggccgccaactttgacctacttaagttggcgggagacgttgagtccaaccctgggccctctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaagaagcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgctgaaacattcaccgagaccatgagttacctatggtcgaacagccagccattcttctgggtccagctgtgtatacccctggccgctgtcatcgttctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaagatgaagcggcatcgataccggtccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaggcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaa

SEQ ID NO: 32 - DNA, 26 bp, synthetic construct
  source 1..26: mol_type = other DNA
atggtccaaactagttcgaagatctc

SEQ ID NO: 33 - DNA, 25 bp, synthetic construct
  source 1..25: mol_type = other DNA
cttggcaaatacgtattcagaatcc

SEQ ID NO: 34 - DNA, 2652 bp, synthetic construct
  source 1..2652: mol_type = other DNA
atggtccaaactagttcgaagatctccggagctcaagatttacacgctatccagcttcaattagaagaagagatgtttaatggtggcattcgtcgcttcgaagcagatcaacaacgccagattgcagcaggtagcgagagcgacacagcatggaaccgccgcctgttgtcagaacttattgcacctatggctgaaggcattcaggcttataaagaagagtacgaaggtaagaaaggtcgtgcacctcgcgcattggctttcttacaatgtgtagaaaatgaagttgcagcatacatcactatgaaagttgttatggatatgctgaatacggatgctacccttcaggctattgcaatgagtgtagcagaacgcattgaagaccaagtgcgcttttctaagctagaaggtcacgccgctaaatactttgagaaggttaagaagtcactcaaggctagccgtactaagtcatatcgtcacgctcataacgtagctgtagttgctgaaaaatcagttgcagaaaaggacgcggactttgaccgttgggaggcgtggccaaaagaaactcaattgcagattggtactaccttgcttgaaatcttagaaggtagcgttttctataatggtgaacctgtatttatgcgtgctatgcgcacttatggcggaaagactatttactacttacaaacttctgaaagtgtaggccagtggattagcgcattcaaagagcacgtagcgcaattaagcccagcttatgccccttgcgtaatccctcctcgtccttggagaactccatttaatggagggttccatactgagaaggtagctagccgtatccgtcttgtaaaaggtaaccgtgagcatgtacgcaagttgactcaaaagcaaatgccaaaggtttataaggctatcaacgcattacaaaatacacaatggcaaatcaacaaggatgtattagcagttattgaagaagtaatccgcttagaccttggttatggtgtaccttccttcaagccactgattgacaaggagaacaagccagctaacccggtgcctgttgaattccaacacctgcgcggtcgtgaactgaaagagatgctatcacctgagcagtggcaacaattcattaactggaaaggcgaatgcgcgcgcctatataccgcagaaactaagcgcggttcaaagtccgccgccgttgttcgcatggtaggacaggcccgtaaatatagcgcctttgaatccatttacttcgtgtacgcaatggatagccgcagccgtgtctatgtgcaatctagcacgctctctccgcagtctaacgacttaggtaaggcattactccgctttaccgagggacgccctgtgaatggcgtagaagcgcttaaatggttctgcatcaatggtgctaacctttggggatgggacaagaaaacttttgatgtgcgcgtgtctaacgtattagatgaggaattccaagatatgtgtcgagacatcgccgcagaccctctcacattcacccaatgggctaaagctgatgcaccttatgaattcctcgcttggtgctttgagtatgctcaataccttgatttggtggatgaaggaagggccgacgaattccgcactcacctaccagtacatcaggacgggtcttgttcaggcattcagcactatagtgctatgcttcgcgacgaagtaggggccaaagctgttaacctgaaaccctccgatgcaccgcaggatatctatggggcggtggcgcaagtggttatcaagaagaatgcgctatatatggatgcggacgatgcaaccacgtttacttctggtagcgtcacgctgtccggtacagaactgcgagcaatggctagcgcatgggatagtattggtattacccgtagcttaaccaaaaagcccgtgatgaccttgccatatggttctactcgcttaacttgccgtgaatctgtgattgattacatcgtagacttagaggaaaaagaggcgcagaaggcagtagcagaagggcggacggcaaacaaggtacatccttttgaagacgatcgtcaagattacttgactccgggcgcagcttacaactacatgacggcactaatctggccttctatttctgaagtagttaaggcaccgatagtagctatgaagatgatacgccagcttgcacgctttgcagcgaaacgtaatgaaggcctgatgtacaccctgcctactggcttcatcttagaacagaagatcatggcaaccgagatgctacgcgtgcgtacctgtctgatgggtgatatcaagatgtcccttcaggttgaaacggatatcgtagatgaagccgctatgatgggagcagcagcacctaatttcgtacacggtcatgacgcaagtcaccttatccttaccgtatgtgaattggtagacaagggcgtaactagtatcgctgtaatccacgactcttttggtactcatgcagacaacaccctcactcttagagtggcacttaaagggcagatggttgcaatgtatattgatggtaatgcgcttcagaaactactggaggagcatgaagagcgctggatggttgatacaggtatcgaagtacctgagcaaggggagttcgaccttaacgaaatcatggattctgaatacgtatttgcc

SEQ ID NO: 35 - DNA, 131 bp, synthetic construct
  source 1..131: mol_type = other DNA
gcctgcaggtcgactctagaggatcctgcgtacagcaatcagttacattgnnnyrnnnyrnnnyrnnnyrnnnyrgatacnnnyrnnnyrnnnyrnnnyrnnnyratggtccaaactagttcgaagatctc

SEQ ID NO: 36 - DNA, 80 bp, synthetic construct
  source 1..80: mol_type = other DNA
cggccagtgaattcgagctcggtacccaatgtaactgattgctgtacgcagataccttggcaaatacgtattcagaatcc

SEQ ID NO: 37 - DNA, 6186 bp, synthetic construct; identical to SEQ ID NO: 27
  source 1..6186: mol_type = other DNA

SEQ ID NO: 38 - DNA, 6168 bp, synthetic construct
  source 1..6168: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgtatggacatattgtcgttagaacgcggctacaattaatacataaccttatgtatcatacacatacgtaattcgactcactatacattggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactgatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccatgaatagaggattctttaacatgctcggccgccgccccttcccagcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcgccaaagcaaccaccgaagccgaagaaaccaaaaacacaggagaagaagaagaagcaacctgcaaaacccaaacccggaaagagacagcgtatggcacttaagttggaggccgacagactgttcgacgtcaaaaatgaggacggagatgtcatcgggcacgcactggccatggaaggaaaggtaatgaaaccactccacgtgaaaggaactattgaccaccctgtgctatcaaagctcaaattcaccaagtcgtcagcatacgacatggagttcgcacagttgccggtcaacatgagaagtgaggcgttcacctacaccagtgaacaccctgaagggttctacaactggcaccacggagcggtgcagtatagtggaggcagatttaccatcccccgcggagtaggaggcagaggagacagtggtcgtccgattatggataactcaggccgggttgtcgcgatagtcctcggaggggctgatgagggaacaagaaccgccctttcggtcgtcacctggaatagcaaagggaagacaatcaagacaaccccggaagggacagaagagtggtctgctgcaccactggtcacggccatgtgcttgcttggaaacgtgagcttcccatgcaatcgcccgcccacatgctacacccgcgaaccatccagagctctcgacatcctcgaagagaacgtgaaccacgaggaattccaagccgccatctcaaaaacttcatggagttggctgtttgcccttttcggcggcgcctcgtcgctattaattataggacttatgatttttgcttgcagcatgatgctgactagcacacgaagatgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaggccggcatggtcccagcctcctcgctggcgccggctgggcaacattccgaggggaccgtcccctcggtaatggcgaatgggacgggaattcctcgagtcttgagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaaataaaacgaaaggctcagtcgaaagactgggcctttcgttttatctgttgtttgtcggtgaacgctctctactagagtcacactggctcaccttcgggtgggcctttctgcggggtttaaactctagatgcattcgcgaggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 39 - DNA, 2686 bp, synthetic construct
  source 1..2686: mol_type = other DNA
gagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaact

SEQ ID NO: 40 - DNA, 11739 bp, synthetic construct
  source 1..11739: mol_type = other DNA
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccagcggccgcatcgatcaccggtcgccaccatgaatagaggattctttaacatgctcggccgccgccccttcccggcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcggccgccaactttgacctacttaagttggcgggagacgttgagtccaaccctgggccctctgctgcaatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcgccgccaactttgacctacttaagttggcgggagacgttgagtccaaccctgggccctctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaagaagcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgctgaaacattcaccgagaccatgagttacctatggtcgaacagccagccattcttctgggtccagctgtgtatacccctggccgctgtcatcgttctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaagatgaagcggcatcgataccggtccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaggcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaa

SEQ ID NO: 41 - DNA, 11739 bp, synthetic construct
  source 1..11739: mol_type = other DNA
attggcggcgtagtacacactattgaatcaaacagccgaccaattgcactaccatcacaatggagaagccagtagttaacgtagacgtagacccgcagagtccgtttgtcgtgcaactgcaaaagagcttcccgcaatttgaggtagtagcacagcaggtcactccaaatgaccatgctaatgccagagcattttcgcatctggccagtaaactaatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgcaagattacgaataagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgcacccggaactatttaccatcaggctatgaaaggcgtgcggaccctgtactggattggcttcgataccacccagttcatgttctcggctatggcaggttcgtaccctgcgtacaacaccaactgggccgacgaaaaagtcctcgaagcgcgtaacatcggactctgcagcacaaagctgagtgaaggcaggacaggaaagttgtcgataatgaggaagaaggagttgaagcccgggtcacgggtttatttctccgttggatcgacactttacccagaacacagagccagcttgcagagctggcatcttccatcggtgttccacctgaaaggaaagcagtcgtacacttgccgctgtgatacagtggtgagctgcgaaggctacgtagtgaagaaaatcaccatcagtcccgggatcacgggagaaaccgtgggatacgcggttacaaacaatagcgagggcttcttgctatgcaaagttaccgatacagtaaaaggagaacgggtatcgttccccgtgtgcacgtatatcccggccaccatatgcgatcagatgaccggcataatggccacggatatctcacctgacgatgcacaaaaacttctggttgggctcaaccagcgaatcgtcattaacggtaagactaacaggaacaccaataccatgcaaaattaccttctgccaatcattgcacaagggttcagcaaatgggccaaggagcgcaaagaagaccttgacaatgaaaaaatgctgggtaccagagagcgcaagcttacatatggctgcttgtgggcgtttcgcactaagaaagtgcactcgttctatcgcccacctggaacgcagaccatcgtaaaagtcccagcctcttttagcgctttccccatgtcatccgtatggactacctctttgcccatgtcgctgaggcagaagataaaattggcattacaaccaaagaaggaggaaaaactgctgcaagtcccggaggaattagtcatggaggccaaggctgctttcgaggatgctcaggaggaatccagagcggagaagctccgagaagcactcccaccattagtggcagacaaaggtatcgaggcagccgcggaagttgtctgcgaagtggaggggctccaggcggacatcggagcagcactcgtcgaaaccccgcgcggtcatgtaaggataatacctcaagcaaatgaccgtatgatcggacagtacatcgttgtctcgccaacctctgtgctgaagaacgctaaactcgcaccagcacacccgctagcagaccaggttaagatcataacgcactccggaagatcaggaaggtatgcagtcgaaccatacgacgctaaagtactgatgccagcaggaagtgccgtaccatggccagaattcttagcactgagtgagagcgccacgctagtgtacaacgaaagagagtttgtgaaccgcaagctgtaccatattgccatgcacggtcccgctaagaatacagaagaggagcagtacaaggttacaaaggcagagctcgcagaaacagagtacgtgtttgacgtggacaagaagcgatgcgtcaagaaggaagaagcctcaggacttgtcctctcgggagaactgaccaacccgccctatcacgaactagctcttgagggactgaagactcgacccgcggtcccgtacaaggttgaaacaataggagtgataggcacaccaggatcgggcaagtcggctatcatcaagtcaactgtcacggcacgtgatcttgttaccagcggaaagaaagaaaactgccgcgaaattgaggccgatgtgctacggctgaggggcatgcagatcacgtcgaagacagtggattcggttatgctcaacggatgccacaaagccgtagaagtgctgtatgttgacgaagcgttcgcgtgccacgcaggagcactacttgccttgattgcaatcgtcagaccccgtaagaaggtagtgctatgcggagaccctaagcaatgcggattcttcaacatgatgcaactaaaggtatatttcaaccacccggaaaaagacatatgtaccaagacattctacaagtttatctcccgacgttgcacacagccagtcacggctattgtatcgacactgcattacgatggaaaaatgaaaaccacaaacccgtgcaagaagaacatcgaaatcgacattacaggggccacgaagccgaagccaggggacatcatcctgacatgcttccgcgggtgggttaagcaactgcaaatcgactatcccggacatgaggtaatgacagccgcggcctcacaagggctaaccagaaaaggagtatatgccgtccggcaaaaagtcaatgaaaacccgctgtacgcgatcacatcagagcatgtgaacgtgctgctcacccgcactgaggacaggctagtatggaaaactttacagggcgacccatggattaagcagctcactaacgtaccaaaaggaaattttcaagccaccatcgaggactgggaagctgaacacaagggaataattgctgcgataaacagtcccgctccccgtaccaatccgttcagctgcaagactaacgtttgctgggcgaaagcactggaaccgatactggccacggccggtatcgtacttaccggatgccagtggagcgagctgttcccacagtttgcagatgacaaaccacactcggccatctacgccctggacgtaatctgcattaagtttttcggcatggacttgacaagcggactgttttccaaacagagcatcccgttaacgtaccatcctgccgattcagcgaggccagtagctcattgggacaacagcccaggaacccgcaagtatgggtacgatcacgccgttgccgccgaactctcccgtagatttccggtgttccagctagctgggaaaggcacacagcttgatttgcagacgggcagaactagagttatctccgcacagcataacttggtcccagtgaaccgcaatctcccgcacgccttagtccccgagcacaaggagaaacaacccggcccggtcaaaaaattcttgagccagttcaaacaccactccgtacttgtggtctcagaggaaaaaattgaagctccccacaagagaatcgaatggatcgccccgattggcatagccggcgctgataagaactacaacctggctttcgggtttccgccgcaggcacggtacgacctggtgtttatcaatattggaactaaatacagaaaccatcactttcagcagtgcgaagaccatgcggcgaccttgaaaaccctctcgcgttcggccctgaactgccttaaccccggaggcaccctcgtggtgaagtcctacggttacgccgaccgcaatagtgaggacgtagtcaccgctcttgccagaaaatttgtcagagtgtctgcagcgaggccagagtgcgtctcaagcaatacagaaatgtacctgatcttccgacaactagacaacagccgcacacgacaattcaccccgcatcatctgaattgtgtgatttcgtccgtgtacgagggtacaagagacggagttggagccgcaccgtcataccgcactaaaagggagaacattgctgattgtcaagaggaagcagttgtcaatgcagccaatccgctgggcagaccaggcgaaggagtctgccgtgccatctataaacgttggccgaacagtttcaccgattcagccacagagaccggcaccgcaaaactgactgtgtgccaaggaaagaaagtgatccacgcggttggccctgatttccggaaacacccagaggcagaagccctgaaattgctgcaaaacgcctaccatgcagtggcagacttagtaaatgaacataatatcaagtctgtcgccatcccactgctatctacaggcatttacgcagccggaaaagaccgccttgaagtatcacttaactgcttgacaaccgcgctagatagaactgatgcggacgtaaccatctactgcctggataagaagtggaaggaaagaatcgacgcggtgctccaacttaaggagtctgtaacagagctgaaggatgaggatatggagatcgacgacgagttagtatggatccatccggacagttgcctgaagggaagaaagggattcagtactacaaaaggaaagttgtattcgtactttgaaggcaccaaattccatcaagcagcaaaagatatggcggagataaaggtcctgttcccaaatgaccaggaaagcaacgagcaactgtgtgcctacatattgggggagaccatggaagcaatccgcgaaaaatgcccggtcgaccacaacccgtcgtctagcccgccaaaaacgctgccgtgcctctgcatgtatgccatgacgccagaaagggtccacagactcagaagcaacaacgtcaaagaagttacagtatgctcctccaccccccttccaaagtacaaaatcaagaacgttcagaaggttcagtgcacaaaagtagtcctgtttaacccgcatacccctgcattcgttcccgcccgtaagtacatagaagcgccagaacagcctgcagctccgcctgcacaggccgaggaggcccccgaagttgcagcaacaccaacaccacctgcagctgataacacctcgcttgatgtcacggacatctcactggacatggaagacagtagcgaaggctcactcttttcgagctttagcggatcggacaactctattactagtatggacagttggtcgtcaggacctagttcactagagatagtagaccgaaggcaggtggtggtggctgacgtccatgccgtccaagagcctgcccctgttccaccgccaaggctaaagaagatggcccgcctggcagcggcaagaatgcaggaagagccaactccaccggcaagcaccagctctgcggacgagtcccttcacctttcttttggtggggtatccatgtccttcggatcccttttcgacggagagatggcccgcttggcagcggcacaacccccggcaagtacatgccctacggatgtgcctatgtctttcggatcgttttccgacggagagattgaggagctgagccgcagagtaaccgagtctgagcccgtcctgtttgggtcatttgaaccgggcgaagtgaactcaattatatcgtcccgatcagccgtatcttttccaccacgcaagcagagacgtagacgcaggagcaggaggaccgaatactgactaaccggggtaggtgggtacatattttcgacggacacaggccctgggcacttgcaaaagaagtccgttctgcagaaccagcttacagaaccgaccttggagcgcaatgttctggaaagaatctacgccccggtgctcgacacgtcgaaagaggaacagctcaaactcaggtaccagatgatgcccaccgaagccaacaaaagcaggtaccagtccagaaaagtagaaaatcagaaagccataaccactgagcgactgctttcagggctacgactgtataactctgccacagatcagccagaatgctataagatcacctacccgaaaccatcgtattccagcagtgtaccggcgaactactctgacccaaagtttgctgtagctgtttgcaacaactatctgcatgagaattacccgacggtagcatcttatcagatcaccgacgagtacgatgcttacttggatatggtagacgggacagtcgcttgcctagatactgcaactttttgccccgccaagcttagaagttacccgaaaagacacgagtatagagccccaaacatccgcagtgcggttccatcagcgatgcagaacacgttgcaaaacgtgctcattgccgcgactaaaagaaactgcaacgtcacacaaatgcgtgaattgccaacactggactcagcgacattcaacgttgaatgctttcgaaaatatgcatgtaatgacgagtattgggaggagtttgcccgaaagccaattaggatcactactgagttcgttaccgcatacgtggccagactgaaaggccctaaggccgccgcactgttcgcaaagacgcataatttggtcccattgcaagaagtgcctatggataggttcgtcatggacatgaaaagagacgtgaaagttacacctggcacgaaacacacagaagaaagaccgaaagtacaagtgatacaagccgcagaacccctggcgaccgcttacctgtgcgggatccaccgggagttagtgcgcaggcttacagccgtcttgctacccaacattcacacgctttttgacatgtcggcggaggactttgatgcaatcatagcagaacacttcaagcaaggtgacccggtactggagacggatatcgcctcgttcgacaaaagccaagacgacgctatggcgttaactggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacatcatacacggagtagtatctgacaaagaaatggctgagaggtgtgccacctggctcaacatggaggttaagatcattgacgcagtcatcggcgagagaccgccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccagcggccgcatcgatcaccggtcgccaccatgaatagaggattctttaacatgctcggccgccgccccttcccggcccccactgccatgtggaggccgcggagaaggaggcaggcggccccgatgcctgcccgcaatgggctggcttcccaaatccagcaactgaccacagccgtcagtgccctagtcattggacaggcaactagacctcaaaccccacgcccacgcccgccgccgcgccagaagaagcaggcggccgccaactttgacctacttaagttggcgggagacgttgagtccaaccctgggccctctgctgcaatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgagcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcgccgccaactttgacctacttaagttggcgggagacgttgagtccaaccctgggccctctgctgcaccactggtcacggccatgtgcttgcttggaaacgtgagcttcccatgcaatcgcccgcccacatgctacacccgcgaaccatccagagctctcgacatcctcgaagagaacgtgaaccacgaggcctacgacaccctgctcaacgccatattgcggtgcggatcgtccggcagaagtaaaagaagcgtcactgacgactttaccttgaccagcccgtacttgggcacatgctcgtactgtcaccatactgaaccgtgctttagcccgattaagatcgagcaggtctgggatgaagcggacgacaacaccatacgcatacagacttccgcccagtttggatacgaccaaagcggagcagcaagctcaaataagtaccgctacatgtcgctcgagcaggatcatactgtcaaagaaggcaccatggatgacatcaagatcagcacctcaggaccgtgtagaaggcttagctacaaaggatactttctcctcgcgaagtgtcctccaggggacagcgtaacggttagtatagcgagtagcaactcagcgacgtcatgcacaatggcccgcaagataaaaccaaaattcgtgggacgggaaaaatatgacctacctcccgttcacggtaagaagattccttgcacagtgtacgaccgtctgaaagaaacaaccgccggctacatcactatgcacaggccgggaccgcacgcctatacgtcctatctggaggaatcatcagggaaagtctacgcgaagccaccatccggaaagaacattacgtacgagtgcaagtgcggcgattacaagaccggtaccgttacgacccgtaccgaaatcacgggctgcaccgccatcaagcagtgcgtcgcctataagagcgaccaaacgaagtgggtcttcaattcgccggacttgatcagacatgccgaccacacggcccaagggaaattgcatttacctttcaagctgatcccgagtacctgcatggtccctgttgcccacgcgccgaacgtagtacacggctttaaacacatcagcctccaattagacacagaccacctgacattgctcaccaccaggagactaggggcaaatccggaaccaactactgaatggatcatcggaaagacggttagaaacttcaccgtcgaccgagatggcctggaatacatatggggcaatcacgaaccggtaagggtctatgcccaagagtctgcaccaggagaccctcacggatggccacacgaaatagtacagcattactaccatcgccatcctgtgtacaccatcttagccgtcgcatcagctgctgtggcgatgatgattggcgtaactgttgcagcattatgtgcctgtaaagcgcgccgtgagtgcctgacgccatatgccctggccccaaatgccgtgattccaacttcgctggcacttttgtgctgtgttaggtcggctaatgctgaaacattcaccgagaccatgagttacctatggtcgaacagccagccattcttctgggtccagctgtgtatacccctggccgctgtcatcgttctaatgcgctgttgctcatgctgcctgccttttttagtggttgccggcgcctacctggcgaaggtagacgcctacgaacatgcgaccactgttccaaatgtgccacagataccgtataaggcacttgttgaaagggcagggtacgccccgctcaatttggagattactgtcatgtcctcggaggttttgccttccaccaaccaagagtacatcacctgcaaattcaccactgtggtcccctcccctaaagtcaaatgctgcggctccttggaatgtcagcccgccgctcacgcagactatacctgcaaggtctttggaggggtgtaccccttcatgtggggaggagcacaatgtttttgcgacagtgagaacagccagatgagtgaggcgtacgtcgaattgtcagcagattgcgcgactgaccacgcgcaggcgattaaggtgcatactgccgcgatgaaagtaggactacgtatagtgtacgggaacactaccagtttcctagatgtgtacgtgaacggagtcacaccaggaacgtctaaagacctgaaagtcatagctggaccaatttcagcatcgtttacaccattcgatcacaaggtcgttatccatcgcggcctggtgtacaactatgacttcccggaatacggagcgatgaaaccaggagcgtttggagacattcaagctacctccttgactagcaaagatctcatcgccagcacagacattagactactcaagccttccgccaagaacgtgcatgtcccgtacacgcaggccgcatctggattcgagatgtggaaaaacaactcaggccgcccactgcaggaaaccgcccctttcgggtgcaagattgcagtcaatccgcttcgagcggtggactgctcatacgggaacattcccatctctatcgacatcccgaacgctgcctttatcaggacatcagatgcaccactggtctcaacagtcaaatgtgatgtcagtgagtgcacttactcagcggacttcggcgggatggctaccctgcagtatgtatccgaccgcgaaggacaatgccctgtacattcgcattcgagcacagcaaccctccaagagtcgacagttcatgtcctggagaaaggagcggtgacagtacacttcagcaccgcgagcccacaggcgaactttattgtatcgctgtgtggtaagaagacaacatgcaatgcagaatgcaaaccaccagctgaccatatcgtgagcaccccgcacaaaaatgaccaagaattccaagccgccatctcaaaaacttcatggagttggctgtttgcccttttcggcggcgcctcgtcgctattaattataggacttatgatttttgcttgcagcatgatgctgactagcacacgaagatgaagcggcatcgataccggtccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaggcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaaaaaaaaaa

SEQ ID NO: 42 - DNA, 21 bp, synthetic construct
  source 1..21: mol_type = other DNA
gccaccatggccaagcctttg

SEQ ID NO: 43 - DNA, 22 bp, synthetic construct
  source 1..22: mol_type = other DNA
cccctgttctcatttccgatcg

SEQ ID NO: 44 - DNA, 114 bp, synthetic construct
  source 1..114: mol_type = other DNA
tcgtcggcagcgtcagatgtgtataagagacaggatacnnnyrnnnyrnnnyrnnnyrnnnyrgatacnnnyrnnnyrnnnyrnnnyrnnnyrgccaccatggccaagcctttg

SEQ ID NO: 45 - DNA, 56 bp, synthetic construct
  source 1..56: mol_type = other DNA
gtctcgtgggctcggagatgtgtataagagacagcccctgttctcatttccgatcg

SEQ ID NO: 46 - DNA, 13998 bp, synthetic construct; identical to SEQ ID NO: 31
  source 1..13998: mol_type = other DNA

SEQ ID NO: 47 - DNA, 2588 bp, synthetic construct
  source 1..2588: mol_type = other DNA
attggcggcgtagtacacactattgaatcaaacagccgaccaattgcactaccatcacaatggagaagccagtagttaacgtagacgtagaccctcagagtccgtttgtcgtgcaactgcaaaagagcttcccgcaatttgaggtagtagcacagcaggtcactccaaatgaccatgctaatgccagagcattttcgcatctggccagtaaactgatcgagctggaggttcctaccacagcgacgattttggacataggcagcgcaccggctcgtagaatgttttccgagcaccagtaccattgcgtttgccccatgcgtagtccagaagacccggaccgcatgatgaaatatgccagcaaactggcggaaaaagcatgtaagattacaaacaagaacttgcatgagaagatcaaggacctccggaccgtacttgatacaccggatgctgaaacgccatcactctgcttccacaacgatgttacctgcaacacgcgtgccgagtactccgtcatgcaggacgtgtacatcaacgctcccggaactatttaccaccaggctatgaaaggcgtgcggaccctgtactggattggcttcgacaccacccagttcatgttctcggctatggcaggttcgtaccctgcatacaacaccaactgggccgacgaaaaagtccttgaagcgcgtaacatcggactctgcagcacaaagctgagtgaaggcaggacaggaaagttgtcgataatgaggaagaaggagttgaagcccatcgcctcgttcgacaaaagccaagacgacgctatggcgttaaccggcctgatgatcttggaagacctgggtgtggaccaaccactactcgacttgatcgagtgcgcctttggagaaatatcatccacccatctgcccacgggtacccgtttcaaattcggggcgatgatgaaatccggaatgttcctcacgctctttgtcaacacagttctgaatgtcgttatcgccagcagagtattggaggagcggcttaaaacgtccaaatgtgcagcatttatcggcgacgacaacattatacacggagtagtatctgacaaagaaatggctgagaggtgtgccacctggctcaacatggaggttaagatcattgacgcagtcatcggcgagagaccaccttacttctgcggtggattcatcttgcaagattcggttacctccacagcgtgtcgcgtggcggaccccttgaaaaggctgtttaagttgggtaaaccgctcccagccgacgatgagcaagacgaagacagaagacgcgctctgctagatgaaacaaaggcgtggtttagagtaggtataacagacaccttagcagtggccgtggcaactcggtatgaggtagacaacatcacacctgtcctgctggcattgagaacttttgcccagagcaaaagagcatttcaagccatcagaggggaaataaagcatctctacggtggtcctaaatagtcagcatagtacatttcatctgactaataccacaacaccaccaccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgaccgctacgccccaatgacccgaccagcaaaactcgatgtacttccgaggaactgatgtgcataatgcatcaggctggtatattagatccccgcttaccgcgggcaatatagcaacaccaaaactcgacgtatttccgaggaagcgcagtgcataatgctgcgcagtgttgccaaataatcactatattaaccatttattcagcggacgccaaaactcaatgtatttctgaggaagcatggtgcataatgccatgcagcgtctgcataactttttattatttcttttattaatcaacaaaattttgtttttaacatttcaaaaaaaaaaaaaaaaaa

SEQ ID NO: 48 - DNA, 100 bp, synthetic construct
  source 1..100: mol_type = other DNA
ctcaaaccccacgcccacgcccgccgccgcgccagaagaagcaggcggccgccaactttgacctacttaagttggcgggagacgttgagtccaaccctgg

SEQ ID NO: 49 - DNA, 100 bp, synthetic construct
  source 1..100: mol_type = other DNA
tacttaagttggcgggagacgttgagtccaaccctgggccctctgctgcaccactggtcacggccatgtgcttgcttggaaacgtgagcttcccatgcaa

SEQ ID NO: 50 - DNA, 100 bp, synthetic construct
  source 1..100: mol_type = other DNA
tacttaagttggcgggagacgttgagtccaaccctgggccctctgctgcaatggtgagcaagggcgaggagctgttcaccggggtggtgcccatcctggt

SEQ ID NO: 51 - DNA, 100 bp, synthetic construct
  source 1..100: mol_type = other DNA
agttcgtgaccgccgccgggatcactctcggcatggacgagctgtacaaggccgccaactttgacctacttaagttggcgggagacgttgagtccaaccc

SEQ ID NO: 52 - DNA, 100 bp, synthetic construct
  source 1..100: mol_type = other DNA
gcgtgcaagattacaaacaagaacttgcatgagaagattaaggatctccgggatcccctgaaaaggctgtttaagttgggtaaaccgctcccagccgacg

SEQ ID NO: 53 - DNA, 100 bp, synthetic construct
  source 1..100: mol_type = other DNA
ccaagccatcagaggggaaataaagcatctctacggtggtcctaaatagtcagcatagtacatttcatctgactaatactacaacaccaccaccatgaat

SEQ ID NO: 54 - DNA, 100 bp, synthetic construct
  source 1..100: mol_type = other DNA
atagcaaagggaagacaatcaagacaaccccggaagggacagaagagtggtctgctgcaccactggtcacggccatgtgcttgcttggaaacgtgagctt

SEQ ID NO: 55 - DNA, 100 bp, synthetic construct
  source 1..100: mol_type = other DNA
gcgaaccatccagagctctcgacatcctcgaagagaacgtgaaccacgaggaattccaagccgccatctcaaaaacttcatggagttggctgtttgccct

SEQ ID NO: 56 - DNA, 100 bp, synthetic construct
  source 1..100: mol_type = other DNA
gacttatgatttttgcttgcagcatgatgctgactagcacacgaagatgatgtttggcccggtggatggctggttagccagagacgggtaagattcctca

SEQ ID NO: 57 - DNA, 100 bp, synthetic construct
  source 1..100: mol_type = other DNA
cctcagggaaggaacaacctaagacaggcacagtcgcagaggggccatcatataccacccgctacgccccaatgacccgaccagcaaaactcgatgtact

SEQ ID NO: 58 - DNA, 100 bp, synthetic construct
  source 1..100: mol_type = other DNA
ctttttattatttcttttattaatcaacaaaattttgtttttaacatttcaaaaaaaaaaaaaaaaaaaaaaaaaagatggccggcatggtcccagcctc

(19) United States
(12) Patent Application Publication
(10) Pub. No.: US 2025/0051796 A1
(43) Pub. Date: Feb. 13, 2025

(54) ENGINEERED VIRAL NUCLEIC ACIDS FOR DIRECTED EVOLUTION AND USES THEREOF

(71) Applicant: Alphinia PTY LTD, Lenah Valley, Tasmania (AU)

(72) Inventor: Minh Thuan Nguyen Tran, South Hobart, Tasmania (AU)

(73) Assignee: Alphinia PTY LTD, Lenah Valley, Tasmania (AU)

(21) Appl. No.: 18/713,598

(22) PCT Filed: Nov. 25, 2022
(86) PCT No.: PCT/AU2022/051417

§ 371 Date: May 24, 2024

(30) Foreign Application Priority Data
Nov. 26, 2021 (AU) 2021903835

(51) Int. Cl. C12N 15/86; C12N 7/00; C12N 15/10

(52) U.S. Cl. CPC C12N 15/86; C12N 7/00; C12N 15/1058; C12N 2770/36122; C12N 2770/36143; C12N 2770/36152; C12N 2800/40

(57) ABSTRACT

The present invention provides nucleic acids sequences, viral particles, viruses, vectors systems, host cells, kits, apparatus, and methods of evolution of a gene product of a gene of interest. The nucleic acid sequences of the invention have been developed primarily for use in evolution of biomolecules of interest. Using a split, non-competent viral vector, the gene of interest can be stably and recombinantly integrated into the viral vector via in-frame insertion of the open reading frame of the gene of interest with an aspect of the native but attenuated viral genome. This configuration of the viral genome, leveraging a split viral vector and an aspect of a viral factor that has been shown to robustly interact with the split viral vector, enables the serial passaging of the recombinant viral particle in an indel and recombination-averse manner.

### FIELD OF THE INVENTION

The present invention provides nucleic acids sequences, viral particles, viruses, vectors systems, host cells, kits, apparatus, and methods of evolution of a gene product of a gene of interest.

The nucleic acid sequences of the invention have been developed primarily for use in evolution of biomolecules of interest and will be described hereinafter with reference to this application. It will, however, be appreciated that the invention is not limited to this particular field of use.

### BACKGROUND OF THE INVENTION

Directed evolution is an effective strategy for developing one or more gene products of a gene of interest with desirable characteristics. During typical directed evolution, a library of genetic variants is established via mutagenesis of an initial gene of interest. Subsequently, expressed gene products of the gene of interest are selected and assayed for a specific activity and/or function.

Known methods of cell culture-based directed evolution using, for example, complementing mutator polymerases suffer from low, non-dynamic mutation rates, and may not produce sufficient mutagenic diversity to effectively explore the available sequence space of an evolving gene of interest. The use of a mutator polymerase with a fixed mutation rate may also suffer from an inability to modulate its mutagenic rate based on the fitness of the evolving gene product without manual experimenter intervention.

In cases wherein attenuated RNA viruses are used with an inserted gene of interest, significant genomic instability and a tendency towards viral recombination occurs. Here, a gene of interest is often inserted into the viral genome, rendering it non-competent in the absence of a viral factor that has been segmented in trans. Viral propagation is therefore dependent on the reconstitution of the complete viral genome to mediate viral egress. Split viral vector systems fail to propagate in an activity-specific manner to the gene of interest, and either fail to produce sufficient viral titres amenable to serial passaging or show loss of system integrity before variants of interest can become fixed in the gene pool. In the former case, the properties of the gene product of the gene of interest have negligible effects on the propagation of the viral particle, as reconstitution of the segmented viral factor fails to efficiently complement the viral particle. In the latter case, loss of system integrity is hallmarked by viral recombination in which the gene of interest becomes unstable and entirely lost from the split viral vector.

The present invention seeks to ameliorate the shortcomings of the known methods of the directed evolution of a gene product of a gene of interest at least partially or to at least provide a useful alternative.

It is to be understood that, if any prior art information is referred to herein, such reference does not constitute an admission that the information forms part of the common general knowledge in the art, in Australia or any other country.

### SUMMARY OF THE INVENTION

There is a need to ameliorate the issue of low and non-dynamic mutation rates by using mutator RNA viruses as a basis for directed evolution and the serial passaging of the gene products thereof. Using a split, non-competent viral vector, the gene of interest can be stably and recombinantly integrated into the viral vector via in-frame insertion of the open reading frame of the gene of interest with an aspect of the native but attenuated viral genome. This configuration of the viral genome, leveraging a split viral vector and an aspect of a viral factor that has been shown to robustly interact with the split viral vector, enables the serial passaging of the recombinant viral particle in an indel and recombination-averse manner. This allows for the steady accumulation of mutations in the gene of interest and allows for its gene products to have improved function and protein solubility. Thus, the present invention discloses a configuration that is amenable to the purposes of directed evolution as sufficiently high viral titres can be achieved without loss of system integrity.

The present invention provides nucleic acid sequences, viral particles, viruses, vectors systems, host cells, kits, apparatus, and methods of evolution of a gene product of a gene of interest.

According to an aspect of the invention there is provided a nucleic acid sequence selected from the group consisting of: a first nucleic acid sequence that includes 5′ to 3′: at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; a second nucleic acid sequence that includes 5′ to 3′: at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence, wherein the 5′ UTR sequence, the one or more non-structural protein(s) coding sequence, the capsid protein coding sequence, the one or more structural protein(s) coding sequence(s), and the 3′ UTR sequence are coding sequences of a Togaviridae virus; and a third nucleic acid sequence that includes 5′ to 3′: at least part of a capsid protein coding sequence, at least part of one or more structural genes coding sequence(s), and at least part of a 3′ UTR sequence, wherein the capsid protein coding sequence, the one or more structural proteins coding sequence(s), and the 3′ UTR sequence are sequences of a Togaviridae virus.

According to another aspect of the invention, there is provided a method of stabilising a recombinant non-competent genome adjacent a 3′ end, the method including inserting a gene of interest in frame with an open reading frame of at least a part of at least one structural protein coding sequence in a non-competent viral vector, wherein the at least one structural protein coding sequence is a Togaviridae virus structural protein coding sequence.

According to another aspect of the invention, there is provided a method of increasing viral titre selected from the group consisting of a first method of increasing viral titre, the first method including: providing a suitable host cell; introducing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are coding sequences of a Togaviridae virus, into the suitable host cell; introducing one or more first complement nucleic acid sequence including 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence, wherein the 5′ UTR sequence, the one or more non-structural protein(s) coding sequence, the capsid protein coding sequence, the one or more structural genes coding sequence(s), and the 3′ UTR sequence are sequences of a Togaviridae virus into the suitable host cell; enabling expression of the viral vector in the suitable host cell; and enabling expression of the nucleic acid sequence in the suitable host cell; and a second method of increasing viral titre, the second method including: providing a suitable host cell; introducing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, and the E1 coding sequence, and the 3′ UTR sequence are sequences of a Togaviridae virus, into the suitable host cell; introducing one or more second complement nucleic acid sequence including 5′ to 3′ at least part of a capsid protein coding sequence, at least part of one or more structural genes coding sequence(s), and at least part of a 3′ UTR sequence, wherein the capsid protein coding sequence, the one or more structural genes coding sequence(s), and the 3′ UTR sequence coding sequences of a Togaviridae virus into the suitable host cell; enabling expression of the viral vector in the suitable host cell; and enabling expression of the nucleic acid sequence in the suitable host cell. It will be appreciated that the order of introduction and expression of the viral vector and the nucleic acid to the suitable host cell may occur in any order or simultaneously.

According to another aspect of the invention, there is provided a method of function-dependent propagation of a Togaviridae virus in a suitable host cell, the method includes providing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; linking expression of the at least part of an in-frame Togaviridae virus capsid nucleic acid sequence and expression of the at least part of one or more in-frame Togaviridae virus structural protein(s) encoding nucleic acid sequence(s) to a function of a gene product of the gene of interest, wherein the at least part of the Togaviridae virus capsid gene and the gene of interest are segmented in trans.

According to any aspect of the invention, there is provided a method of evolution of a gene product of a gene of interest, the method including: providing one or more viral vector(s) comprising 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; providing one or more first complement nucleic acid sequence(s) comprising 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence into the population of suitable host cells, wherein the 5′ UTR sequence, the one or more non-structural protein(s) coding sequence, the capsid protein coding sequence, the one or more structural genes coding sequence(s), and the 3′ UTR sequence are sequences of a Togaviridae virus, and wherein expression of the one or more first complement nucleic acid sequence(s) is linked to a function of a gene product of the gene of interest; introducing the one or more viral vector(s) to a population of suitable host cells; introducing the one or more first complement nucleic acid sequence(s) to the population of suitable host cells; allowing maturation and egress of one or more mature virus(es) that comprise the gene of interest or a variant thereof from the population of suitable host cells; recovering the one or more mature virus(es); introducing the one or more mature virus(es) and one or more first complement nucleic acid sequence(s) into a population of naïve suitable host cells; allowing further maturation and egress of further one or more mature virus(es) that comprise the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s).

Another aspect of the invention provides a method of evolution of a gene product of a gene of interest selected from the group consisting of: a first method of evolution of a gene product of a gene of interest, the first method including: providing one or more viral vector(s) including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; providing one or more first complement nucleic acid sequence(s) including 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence into the population of suitable host cells, wherein the 5′ UTR sequence, the one or more non-structural protein(s) coding sequence, the capsid protein coding sequence, the one or more structural genes coding sequence(s), and the 3′ UTR sequence are sequences of a Togaviridae virus, and wherein expression of the one or more first complement nucleic acid sequence(s) is linked to a function of a gene product of the gene of interest; introducing the one or more viral vector(s) to a population of suitable host cells; introducing the one or more first complement nucleic acid sequence(s) to the population of suitable host cells; allowing maturation and egress of one or more mature virus(es) that include the gene of interest or a variant thereof from the population of suitable host cells; recovering the one or more mature virus(es); introducing the one or more mature virus(es) and one or more first complement nucleic acid sequence(s) into a population of naïve suitable host cells; allowing further maturation and egress of further one or more mature virus(es) that include the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s); and a second method of evolution of a gene product of a gene of interest, the second method including: providing one or more viral vector(s) including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, and the E1 coding sequence, and the 3′ UTR sequence are sequences of a Togaviridae virus; providing one or more second complement nucleic acid sequence(s) including 5′ to 3′ at least part of a capsid protein coding sequence, at least part of one or more structural genes coding sequence(s), and at least part of a 3′ UTR sequence, wherein the capsid protein coding sequence, and the one or more structural genes coding sequence(s), and the 3′ UTR sequence are sequences of a Togaviridae virus, and wherein expression of the one or more second complement nucleic acid sequence(s) is linked to a function of a gene product of the gene of interest; introducing the one or more viral vector(s) to a population of suitable host cells; introducing the one or more complement nucleic acid(s) to the population of suitable host cells; allowing maturation and egress of one or more mature virus(es) that include the gene of interest or a variant thereof from the population of suitable host cells; recovering the one or more mature virus(es); introducing the one or more mature virus(es) and one or more second complement nucleic acid sequence(s) into a population of naïve suitable host cells; allowing further maturation and egress of further one or more mature virus(es) that include the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s). It will be appreciated that the steps may be repeated to further evolve the gene product of the gene of interest. It will also be appreciated that the order of introducing the one or more viral vector(s) to a population of suitable host cells and introducing the one or more first and/or second complement nucleic acid(s) to a population of suitable host cells may occur in any order or simultaneously.

In some embodiments, the Togaviridae virus is an Alphavirus. In some embodiments, the Alphavirus is Sindbis virus.

### BRIEF DESCRIPTION OF THE DRAWINGS

Notwithstanding any other forms which may fall within the scope of the present invention, a preferred embodiment/preferred embodiments of the invention will now be described, by way of example only, with reference to the accompanying drawings in which:

FIG. 1 is a schematic overview outlining a mechanism of action for an RNA biosensor according to the invention. Modified SinCapsid mRNA complement (SINV biosensor) containing an mCherry fluorophore transgene downstream from a 26S promoter strongly expresses the mCherry fluorophore within 4 hpi. SINV biosensor mRNA is introduced either simultaneously with viral mRNA components via nucleofection at 0 hpi, or via lipid transfection 1 hpi following application of Sindbis viral particles to naive BHK-21 cells. mCherry signal appears inversely proportional to the formation of replication complexes (RC), which could preclude interaction between the SINV biosensor RNA complement and the Sindbis virus non-structural genes (nsp1-4) required to induce transcription from the 26S promoter. SINV, Sindbis virus; RdRp, RNA-dependent RNA polymerase (nsp1-4 complex); RC, replication complex; red barrel shapes objects, mCherry.

FIG. 2 is a graphical representation of results obtained depicting utility of the RNA biosensor. Strongest interaction between Sindbis viral particles and RNA biosensor complement occurs upon cointroduction or after one hour post infection (hpi).

FIG. 3 is a schematic representation of a genomic arrangement of split Sindbis viral nucleic acid sequences which have had either the capsid gene fragment, or the structural glycoproteins removed. A gene-of-interest (GOI) is inserted either downstream of a secondary sub-genomic promoter (dsSIN Capsid or dsSIN Envelope) or inserted in-frame with the capsid gene (SIN2A Capsid). A 2A protease signal derived from foot-and-mouth disease virus (FMD) was used downstream of the GOI to separate it from other aspects of the structural proteins.

FIG. 4 is a schematic representation of passaging steps according to the invention. Schematic showing the passaging method for the determination of cytopathic effects (CPE) indicating viral recombination and competency. Viral variants were passaged serially for five rounds under genetic drift in which the complement component of the recombinant virus is provided in trans, and two rounds of ‘orthogonal passaging’ on naïve cells at the exclusion of their complement components.

FIG. 5 is a heatmap showing non-cytopathic-effects-(CPE)-mediated recombination and competency for dsSIN-Envelope and SIN2A-Capsid, which shows robust GFP expression on the second round of orthogonal passaging as a ratio of GFP+ to brightfield-imaged cells according to the invention. A heatmap showing non-CPE-mediated recombination and competency for dsSIN-Envelope and SIN2A-Capsid, which shows robust GFP expression on the second round of orthogonal passaging as a ratio of GFP+ to brightfield-imaged cells.

FIG. 6 is a representative heatmap displaying the CPE of four viral redesigns following serial passaging, except for an dsSIN-Envelope condition, according to the invention. Representative heatmap displaying the CPE of the four viral redesigns following serial passaging, except for the dsSIN-Envelope condition.

FIG. 7 is schematic representation of engineering of a 26S sub-genomic region of an SIN2A-Envelope variant according to the invention. Engineering of the 26S sub-genomic region of the SIN2A-Envelope variant.

FIG. 8 is a heatmap that depicts effects of removing a packaging signal (PS) from the complement component intended for an SIN2A-Envelope viral variant, as well as a negligible CPE observed during serial passaging upon provision of a complement from a plasmid according to the invention. The heatmap displays the effects of removing the packaging signal (PS) from the complement component intended for the SIN2A-Envelope viral variant, as well as the negligible CPE observed during serial passaging upon provision of the complement from a plasmid. The plasmid expresses the proteinaceous capsid with no 5′UTR or PS elements.

FIG. 9 is heatmap that depicts effects of deletion of a partial capsid fragment results in robust transgene expression but mediates persistent, non-CPE indicating viral recombination and competency according to the invention. Deletion of the partial capsid fragment results in robust transgene expression but mediates persistent, non-CPE indicating viral recombination and competency. At round 3 of orthogonal passaging in the absence of the viral complement, GFP foci are evident, as measured by the ratio of GFP+ cells to a brightfield imaged cells for the same field of view.

FIG. 10 is an image of representative wells of a plaque assay performed with associated GFP images of recombinant viral redesigns for indicated passage round. Representative wells of the plaque assay performed with their GFP images of the recombinant viral redesigns for indicated passage round.

FIG. 11 is a graphical representation of mutation frequency as determined across five rounds of serial passaging under genetic drift conditions according to the invention. Variants were called using LoFreq and setting a minimal allele frequency (MAF) of 0.0025 followed by the subtraction of background sequencing and PCR errors as determined by “Plasmid” sample of the SIN2A-Envelope, which underwent the same pipeline. Mutation frequency is reported as nucleotide mutation per base pair per round of passaging.

FIG. 12 is a graphical representation depicting an SIN2-envelope construct used to screen for nonsense mutations according to the invention. Blasticidin resistance gene (BSR) was inserted into the SIN2A-Envelope construct with and without the introduction of a stop codon (C56X).

FIG. 13 is a graphical representation of passaging steps under permissive, genetic drift conditions for three rounds using mRNA transfection according to the invention. Viral variants were passaged under permissive, genetic drift conditions for three rounds using mRNA transfection. Briefly, R0 (Round 0) virus was applied at an approximate MOI of 1 following titration of viral variants using qPCR. Subsequent passages (R1, R2, and R3; Round 1, Round 2, and Round 3, respectively) were applied by taking 1 mL of clarified, undiluted virus from the previous passage and applying the viral supernatant to BHK-21 cells.

FIG. 14 is a graphical representation of selection of a SIN2A-Envelope variant of SINV against introduction of nonsense mutations in an open reading frame (ORF) of a gene-of-interest (GOI) in accordance with Muller's Ratchet principle according to the invention. Viral variants containing the full BSR ORF were serially propagated with amplifying viral titres, whereas the C56X variant was unable to recover due to disruption of the structural glycoprotein ORF (n=2). NTC, no template control.

FIG. 15 is a graphical representation of preliminary testing of an SP6 circuit; SP6 RNA polymerase (SP6 RNAP), which natively accepts an SP6 promoter, expressed under a CMV promoter following transfection in naive BHK-21 cells according to the invention. Preliminary testing of the SP6 circuit; SP6 RNA polymerase (SP6 RNAP), which natively accepts an SP6 promoter, was expressed under a CMV promoter following transfection in naive BHK-21 cells. A cytosolic plasmid (pUC57-SP6-mCherry) bearing an SP6 promoter driving an mCherry fluorophore was co-transfected and fluorescence was observed.

FIG. 16 is a graphical representation of recombinant SP6 RNAP incubated with a complement plasmid expressing SinCapsid under an SP6 promoter, SP6/T7 hybrid promoter, or a T7 promoter for one hour and visualized on a 1% agarose gel to confirm loss of activity on a non-native SP6 promoter in vitro according to the invention. Recombinant SP6 RNAP was incubated with the complement plasmid expressing SinCapsid under either an SP6 promoter, SP6/T7 hybrid promoter or a T7 promoter for one hour and visualized on a 1% agarose gel to confirm loss of activity on a non-native SP6 promoter in vitro.

FIG. 17 is a schematic representation of a SIN2A-Envelope expressing mutagenized SP6 RNA polymerase (SIN2A-SP6-Envelope) serially passaged on a transfected BHK-21 cell line expressing a viral complement under a modified T7 promoter (TC86-T7) according to the invention. SIN2A-Envelope expressing mutagenized SP6 RNA polymerase (SIN2A-SP6-Envelope) was serially passaged on a transfected BHK-21 cell line expressing the viral complement under a modified T7 promoter (TC86-T7). SP6 RNAP natively accepts an SP6 promoter at round 5 of serial passaging, an increase in viral titre as measured by qPCR against the E2 glycoprotein was observed, which increased progressively until cytopathic effect (CPE) was observed at round 7.

FIG. 18 is a graphical representation of successive rounds of serially passaged SIN2A-SP6-Envelope, which results in enrichment of mutations throughout an open reading frame of SP6 according to the invention. Mutations are mapped for viral variants serially passaged under permissive, genetic drift conditions, along with those mutations mapped following two and seven rounds of selection (R3R2R2). Representative amino acids are coloured and mapped onto the surface representation of the predicted structure for SP6 RNAP as determined by AlphaFold2. Active site (AS) residues are represented in orange.

FIG. 19 is a schematic representation of an SIN2A-Envelope polynucleotide according to the invention. Virus design showing the simplified construct map of recombinant, split Sindbis virus (SIN2A-Envelope). A gene-of-interest (GOI) is inserted in-frame between partial capsid fragment and the complete open reading frame of structural glycoproteins (E3, E2, 6K, E1) intervened by bipartite 2A protease cleavage signal from foot-and-mouth disease virus (FMD).

FIG. 20 is a schematic representation of an SIN2A-Envelope complement polynucleotide in a relaxed form and a stringent form according to the invention. Circuitous amplification of the SIN2A-Envelope construct is dependent on the provision of the in trans complement, of which two circuits are possible. The ‘relaxed’ circuit permits some degree of co-packaging between the complement and the SIN2A-Envelope component owing to the inclusion of the packaging signal (PS), whereas the ‘stringent’ circuit almost entirely abrogates co-packaging and places a higher selective burden for circuitous amplification.

FIG. 21 is a schematic representation of a Capsid expression in a relaxed circuit according to the invention. Relaxed circuit leveraging the expression of the RNA complement from a cytosolic pUC19 plasmid expressing RNA (IncRNA) from a T7 promoter. Induction of the T7 promoter produces the RNA complement for SIN2A-Envelope, enabling interaction between the in trans 26SP from the complement and the RdRp component of SIN2A-Envelope. Refractive amplification enables productive expression of the proteinaceous capsid component, complementing the split virus and allowing for viral egress. 5′UTR, 5′ untranslated region; nsp1, non-structural gene 1; nsp4, non-structural gene 4; 26SP, 26 sub-genomic promoter; SL1234, stem loops 1,2,3,4; 3′UTR, 3′ untranslated region; HDV ribozyme, Hepatitis delta virus ribozyme; and pA, polyA signal.

FIG. 22 is a schematic representation of a circuitous amplification of a split Sindbis virus according to the invention. BHK-21 cells can be engineered to express recombinant T7 RNA polymerase (RNAP) and amplification of the split Sindbis virus (SIN2A-Envelope) is achieved by coupling the expression of the in trans complement with a gated selection circuit.

FIG. 23 is a schematic representation disruption and restoration of a T7 RNA polymerase (RNAP) according to the invention. One-hybrid circuit leveraging the restoration of the open reading frame (ORF) of T7 RNAP drives the expression of the complement allowing for interaction with the non-structural genes of the SIN2A-Envelope component. Disruption of the ORF of T7 requires correction that can be enabled by a packaged gene-of-interest (GOI) in the SIN2A-Envelope, which accumulates necessary mutations to impart improved functionality.

FIG. 24 is a schematic representation of circuitous amplification of split SINV virus (SIN2A-Envelope) achieved by coupling expression of the complement polynucleotide of FIG. 1.2 with activation of a tetracycline-transactivator (tTa) RNA polymerase (RNAP) according to the invention. Engineered BHK-21 cells were lentivirally integrated with tTa. Expression of tTA induces the expression of the viral complement under the ‘strict’ circuit in which proteinaceous capsid is expressed for nucleocapsid assembly for viral egress. Open reading frame (ORF) of tTa is disrupted; introduction of SIN2A-Envelope carrying a gene-of-interest (GOI) capable of reverting the disrupted ORF enables expression of tTa, which drives expression of capsid complement from a nuclear-localized plasmid.

FIG. 25 is a schematic representation of viral entry into and egress from a host cell in an activity gated manner according to the invention. Expression of capsid complement facilitates nucleocapsid assembly, which enables viral budding. Relevant mutations in the GOI meant to enhance the reversion of the disrupted tTa ORF accumulates and is packaged as part of viral genome encapsidation prior to viral egress.

FIG. 26 is a general overview of a use of a viral vector in circuitous evolution of a gene of interest according to the invention. A construct expressing the SIN2A-Envelope viral variant containing ae gene-of-interest (GOI) undergo randomized in vitro mutagenesis using GOI-specific primers and subsequent transformation and selection in competent E. coli cells. Extraction of mutagenic library is followed by appropriate linearization and in vitro transcription, followed by purification of in vitro transcribed mRNA. Mutagenized mRNA is nucleofected with the complement component enabling production of Sindbis viral particles that undergo iterative serial passaging in a circuitous manner. Viral supernatant is harvested and the GOI is amplified via RT-PCR and gel extracted. UMI-specific primers assign randomized nucleotides to the gel-extracted GOI to assign a unique molecular barcode to each PCR amplicon allowing for the elimination of background sequencing artefacts associated with nanopore sequencing.

FIG. 27 is a graphical representation and photomicrographs of a split, recombinant Sindbis virus containing an EGFP transgene reporter (pTSIN-EGFP) generated using nucleofection of in vitro transcribed SinCapsid, SinHelper, and pTSIN-EGFP mRNA, as described using the VEGAS platform. In total, five conditions were evaluated for their capacity to enable the serial passaging of the VEGAS system under genetic drift condition. Briefly, 250,000 BHK-21 cells were plated in a T25 flask, and transfected using pCMV-SSG, which is a mammalian expression plasmid enabling the overexpression Sindbis virus structural genes and envelope proteins (Capsid, E3, E2, 6K and E1, respectively) under a constitutive CMV promoter. The transfected cells were subsequently transduced with undiluted viral supernatant generated from the previous round, and after 24 hours, the cells were observed for viral transduction efficiency using EGFP fluorescence as a reporter. As compared to RNA transfection of cells in which in vitro transcribed SinCapsid and SinHelper mRNA were introduced, a significant drop in viral titre was observed as measured by the number of EGFP foci within a limited field of view. As pCMV-SSG contains no upstream elements to the structural genes, such as 5′UTR or non-structural genes, three conditions were performed in parallel in which in vitro transcribed SinCapsid and SinHelper had their upstream 5′ elements removed from one or both of the IncRNAs to evaluate the necessity of these 5′ upstream elements to enable serial passaging of the split, recombinant Sindbis virus. Deletion of these upstream elements showed that they were necessary for sufficient viral propagation of a split, recombinant Sindbis virus under genetic drift conditions.

FIG. 28 shows the tetracycline transactivator (tTA) open reading frame integrated into the SIN2A-vector and evolved against varying concentrations of doxycycline and defective interfering particles across several rounds. Variations to the circuit set-up in which the induction of the tTA-inducible PspCas13b, which knockdowns defective interfering RNA, were tested in a plasmid set-up (A, B) and in a cell line set-up (C, D). Mutational profile was evaluated with variations to the concentration of defective interfering RNA spiked into each round, with stringent (A, C, D) and relaxed selective (B) pressures tuned with respect to low multiplicity of infection (A, C) and high multiplicity of infection (B, D). The mutational landscape revealed the prevalence of several putative mutations that conferred doxycycline insensitivity that did not occur in the initial round 0 virus, or in the genetic drift condition in the absence of doxycycline addition (Negative).

FIG. 29 is a graphical representation relating to the characterisation of several mutants identified from several rounds of evolving a doxycycline-resistant mutant of a tetracycline transactivator (tTA) protein incorporated into the SIN2A-vector. Single nucleotide mutations enriched in the non-genetic drift conditions were re-synthesised into a wildtype pCMV-tTA plasmid upon identification of mutants following next generation deep sequencing of the viral supernatant derived from several trials of selection. Next, the mutant variants of tTA were incubated in the presence or absence of 100 nM of doxycycline to evaluate the induction of an mCherry reporter construct driven by a TETO7 promoter. Apart from some mutants, the induction of tTA is mostly unchanged in the absence of doxycycline. However, when media containing 100 nM of doxycycline is supplemented, several mutants show significant induction of the mCherry reporter relative to the wildtype variant of tTA, with modest induction evident by several other mutants.

FIG. 30 shows the tetracycline transactivator (tTA) open reading frame integrated into the SIN2A-vector (SIN2A-tTA-ENV) and evolved against varying concentrations of doxycycline, which was supplemented into the media (2 nM DOX, 20 nM DOX, 200 nM DOX, 2 μM DOX, and 20 μM DOX). Briefly, per T25 flask, 250,000 BHK21 cells were plated and equimolar amounts of defective helper (DH) and defective interfering (DIP) RNA were supplemented in the presence of viral supernatant from the preceding round. Viral supernatant containing the SIN2A-tTA-ENV recombinant viral particles were transduced at a multiplicity of infection of 1, and qPCR was performed tiled against the packaging signal (PS), which provides a global measure of selection strength, and the viral glycoprotein (E2), which provides a measure of viral particles.

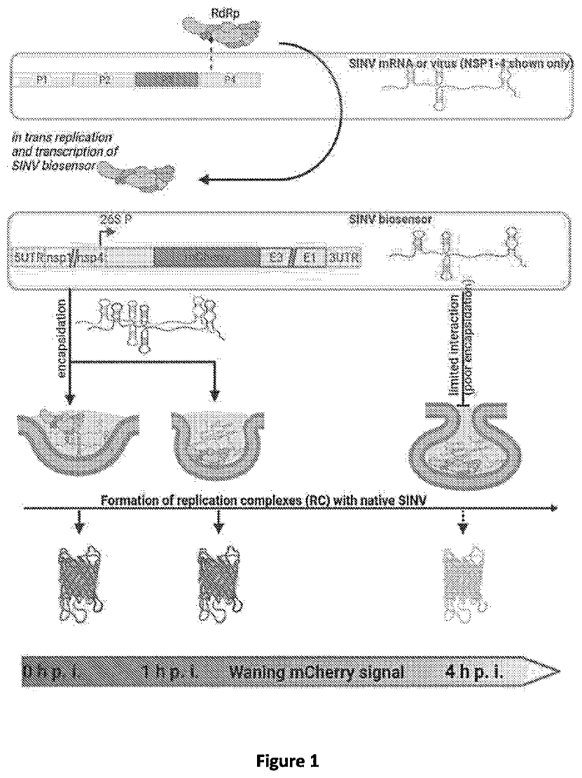

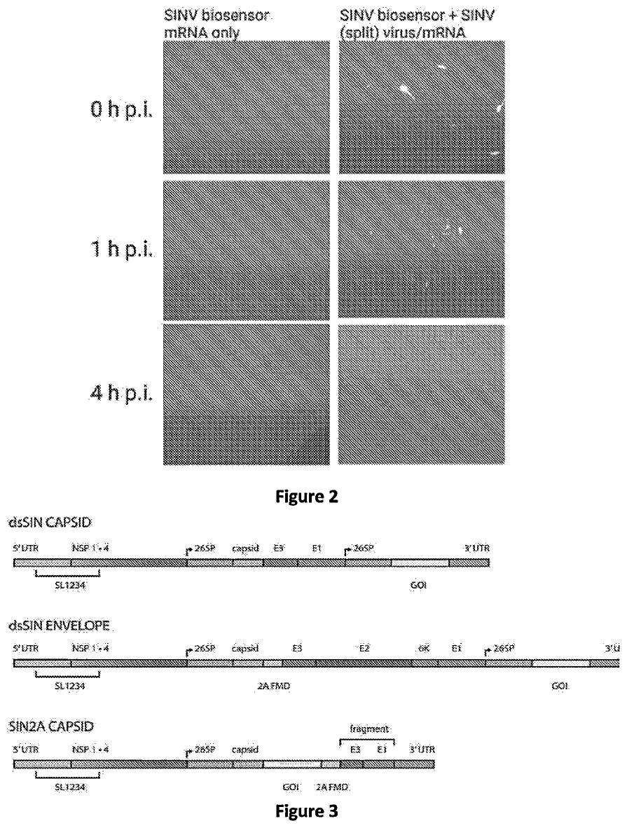

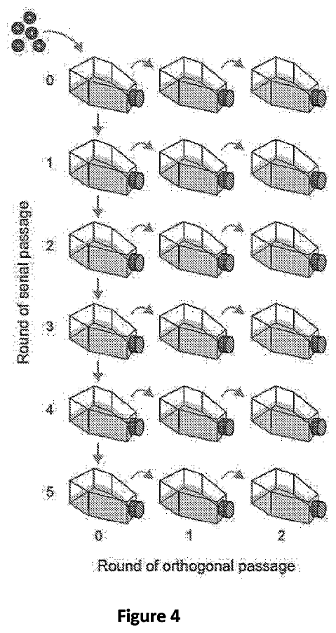

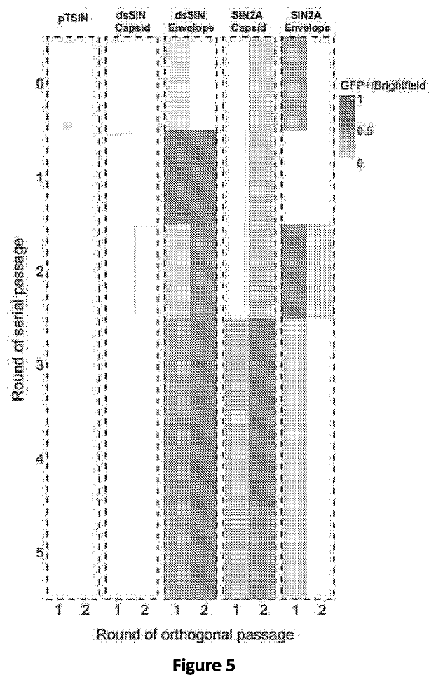

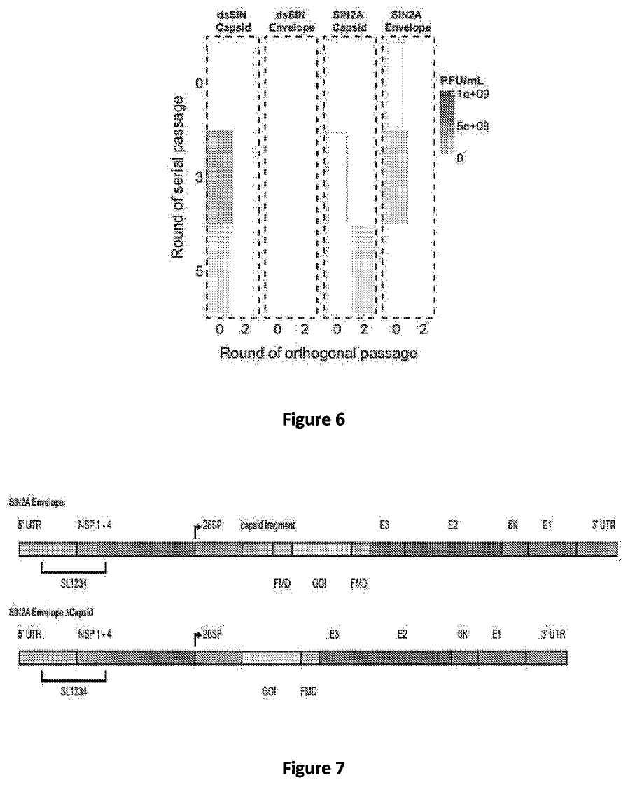

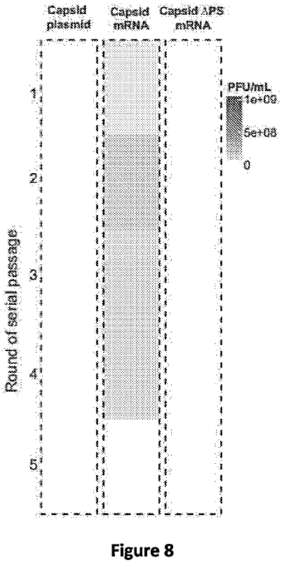

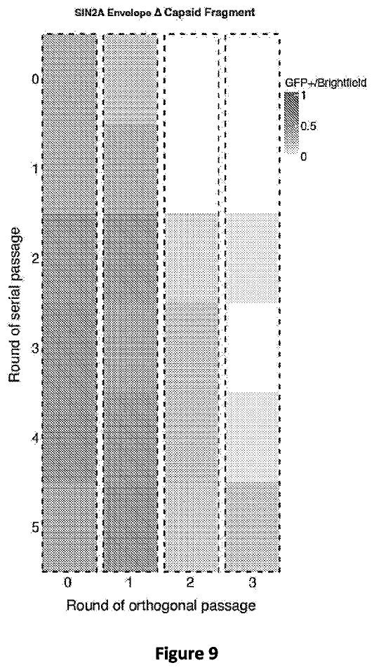

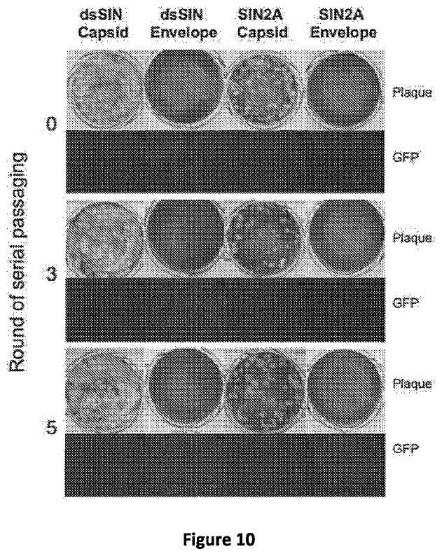

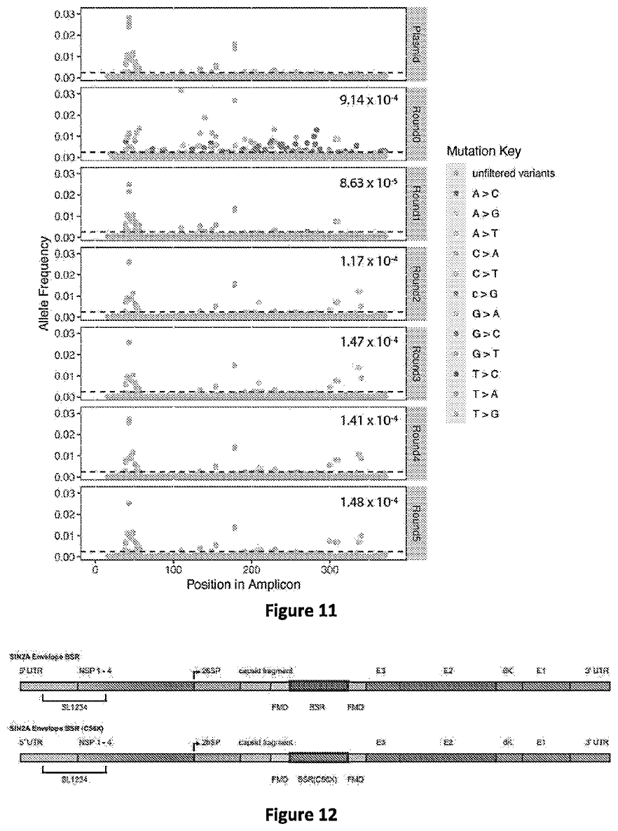

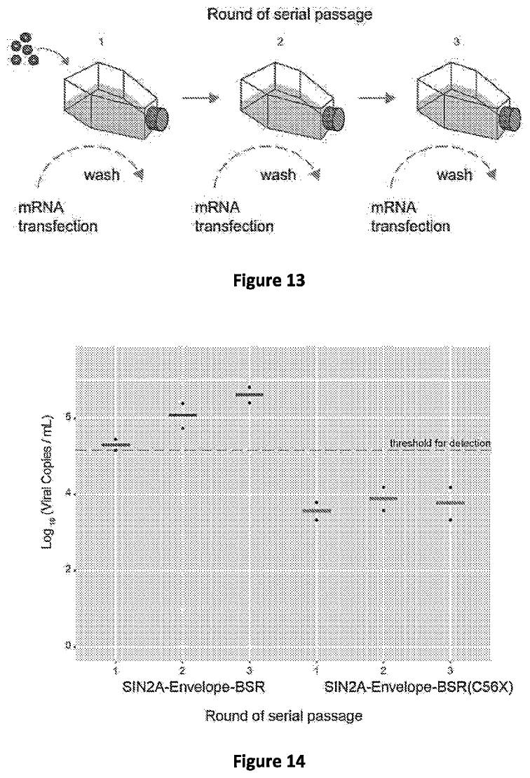

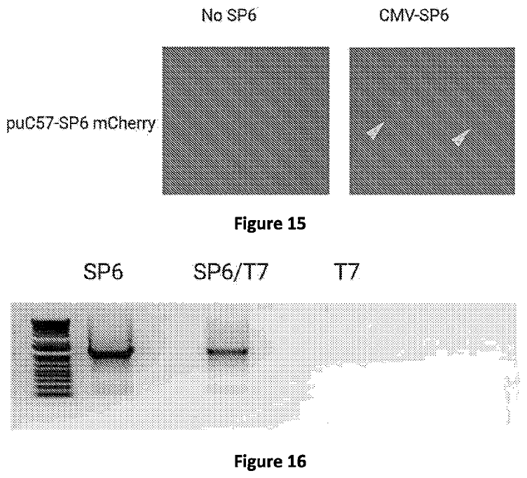

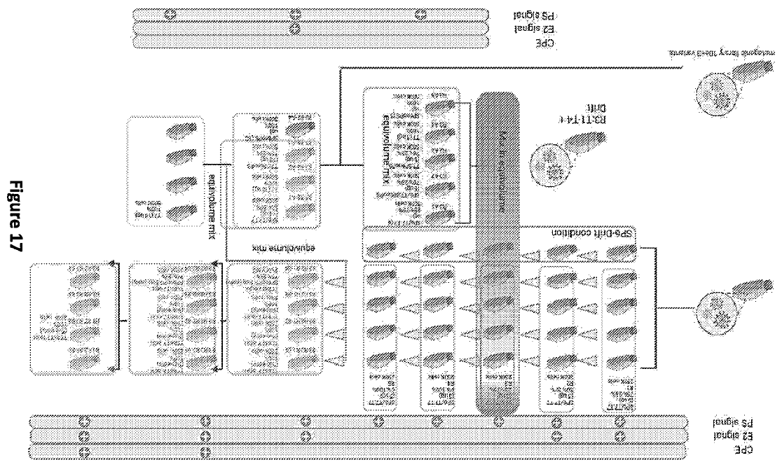

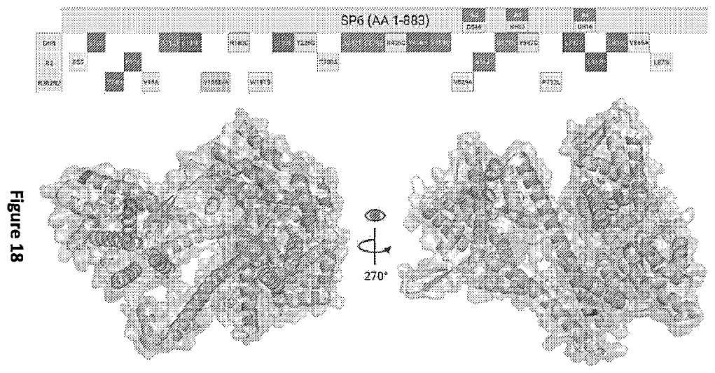

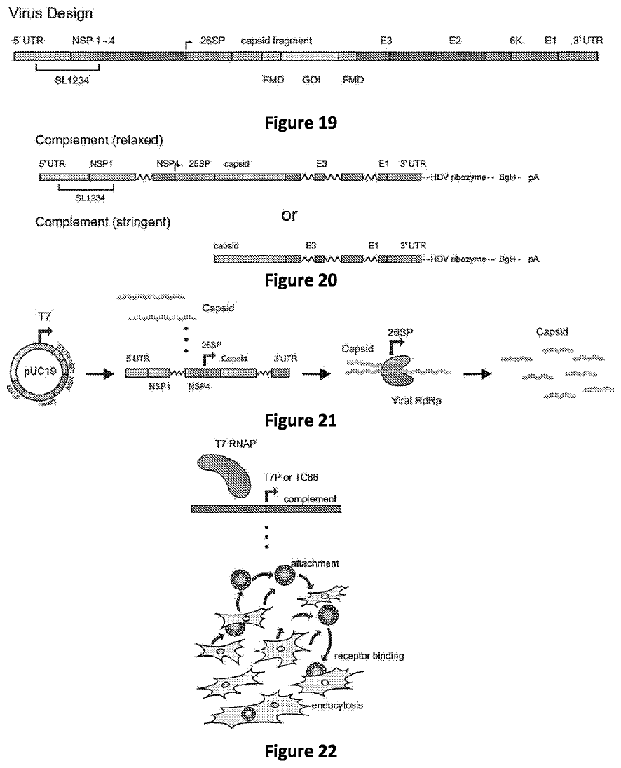

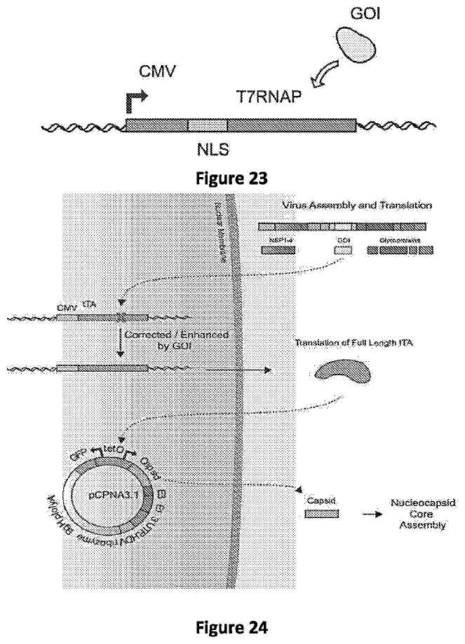

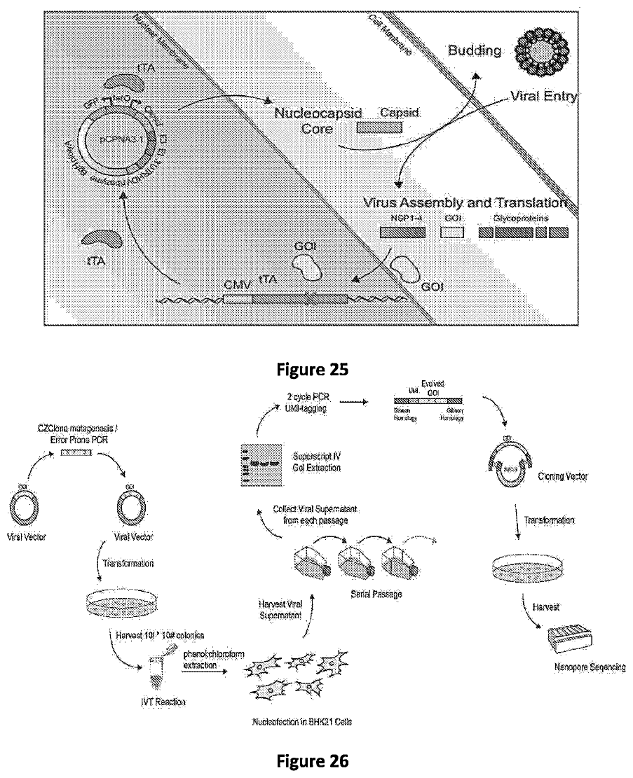

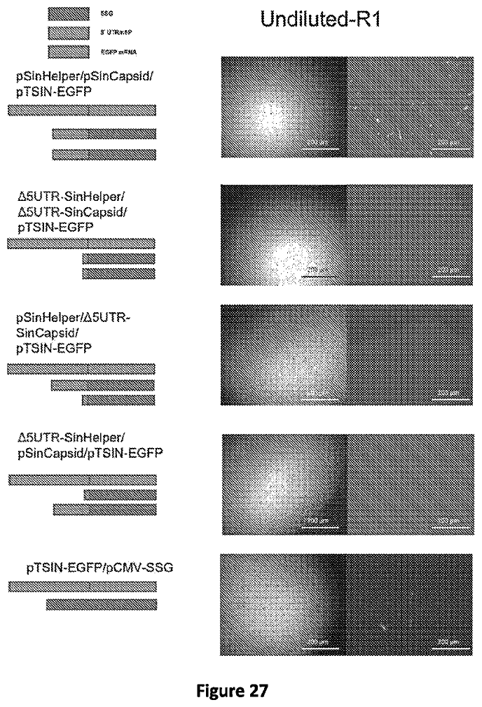

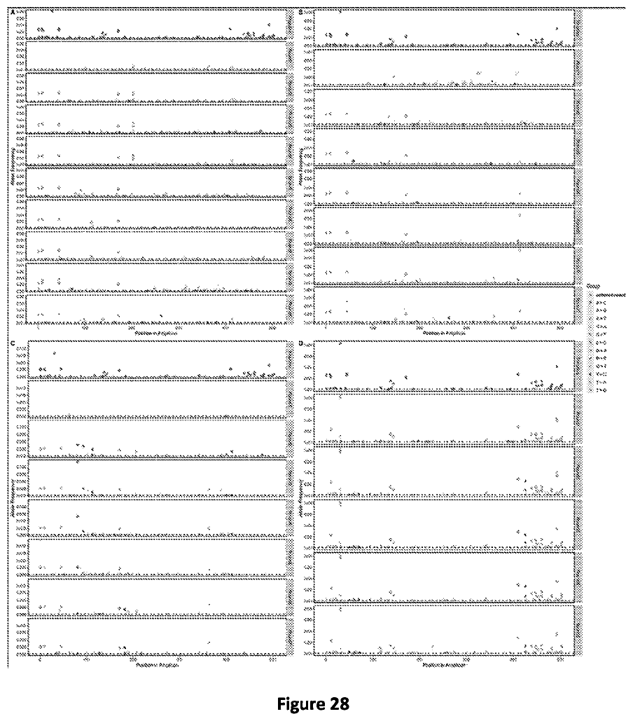

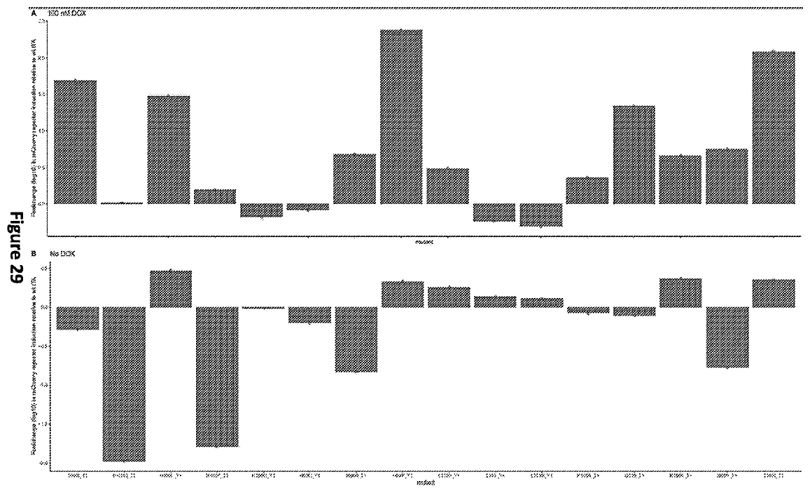

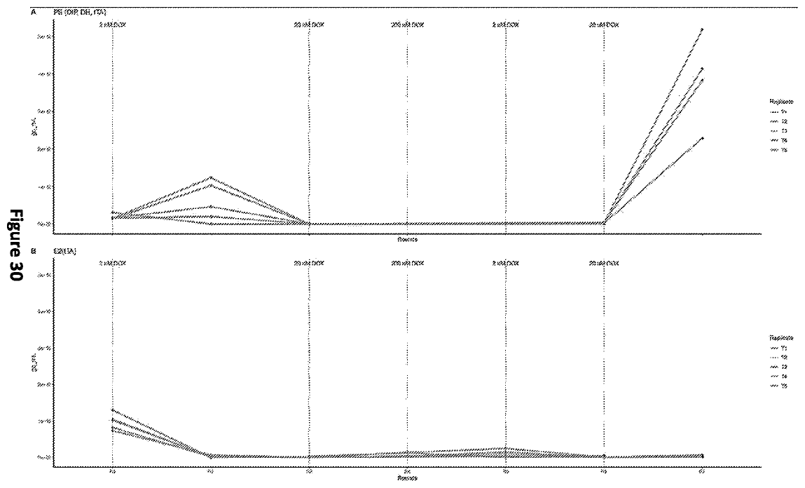

### DESCRIPTION OF EMBODIMENTS

Notwithstanding any other forms which may fall within the scope of the present invention, preferred embodiments of the invention will now be described, by way of example only, with reference to the accompanying figures, as relevant.

The present invention employs methods and techniques that are known to a person having ordinary skill in the art. Such methods and techniques as employed in the present invention include conventional molecular biology, microbiology, and recombinant DNA methods and techniques as disclosed and explained fully in the relevant literature, for example only, Becker's World of the Cell, 9^{th} edition, Hardin, J., et al., Pearson (2015); Essential Cell Biology, 5^{th} edition, Alberts, B, et al., T&F/Garland (2019); Freshney's Culture of Animal Cells: A Manual of Basic Technique and Specialized Applications, Freshney, R. I., and Capes-Davis, A., Wiley-Blackwell (2021); Gel Electrophoresis: Nucleic Acids, Martin, R., Garland Science (2020); Karp's Cell and Molecular Biology, 9^{th} edition, Karp, G., et al., Wiley (2020); Lewin's Genes, 12^{th} edition, Krebs, J. E., et al., Jones & Bartlett Learning (2017); Molecular Biology of the Cell, 6^{th} edition, Alberts, B., et al., Garland Science (2014); Molecular Biology of the Gene, 7^{th} edition, Watson, J., et al., Pearson (2013); Molecular Biology, 5^{th} edition, Weaver, R., McGraw-Hill Education (2011); Molecular Biology: Principles of Genome Function, 2^{nd} edition, Craig, N., et al., Oxford University Press (2014); Molecular Cell Biology, 8^{th} edition, Lodish, H, W. H. Freeman (2016): Molecular Cloning: A Laboratory Manual, Volumes 1, 2, and 3, 4^{th} edition, Green, M. R., and Sambrook, J., Cold Spring Harbour Laboratory Press (2014); and Nucleic Acid Hybridization, Anderson, M. L. M., Garland Science (2020).

Abbreviations of amino acids and nucleic acids, and analogs and derivatives of amino acids and nucleic acids will be known to a person having ordinary skill in the art. Such abbreviations may be found as published as the International Union of Pure and Applied Chemistry (IUPAC) and the International Union of Biochemistry and Molecular Biology (IUBMB) recommendation (see, for example only, Amino Acids and Peptides, 1985, 16, 387-410; Arch. Biochem. Biophys. 1971, 145, 425-436; Biochem. J., 1971, 120, 449-454; Biochem. J., 1984, 219, 345-373; Biochem. J., 1985, 229, 281-286; Biochemical Nomenclature and Related Documents, 2nd edition, Portland Press, 1992, pages 39-69, 109-114, and 122-126; Biochemistry, 1971, 9, 4022-4027; Biochim. Biophys. Acta 1971, 247, 1-12; Eur. J. Biochem., 1970, 15, 203-208; 1972, 25, 1; Eur. J. Biochem., 1984, 138, 9-37; 1985, 152, 1; 1993, 213, 2; Eur. J. Biochem., 1985, 150, 1-5; Internat. J. Pept. Prot. Res., 1984, 24, following p 84; J. Biol. Chem., 1970, 245, 5171-5176; J. Biol. Chem., 1985, 260,14-42; J. Biol. Chem., 1986, 261, 13-17; J. Mol. Biol., 1971, 55, 299-310; Mol. Biol. Evol., 1986, 3, 99-108; Nucl. Acids Res., 1985, 13, 3021-3030; Proc. Nat. Acad. Sci. (U. S.), 1986, 83, 4-8; Pure Appl. Chem., 1974, 40, 277-290; and Pure Appl. Chem., 1984, 56, 595-624 with respect to abbreviations of this nature.

The terms “biomolecule”, “biomacromolecule”, and “biological macromolecule” as used herein should be understood to refer to naturally occurring macromolecular compounds and synthetic macromolecular compounds such as nucleic acids, proteins, peptides, lipids, and carbohydrates.

Nucleic acids, also termed “polynucleotides”, as used herein should be understood to include deoxyribonucleic acid (DNA), ribonucleic acid (RNA), artificial nucleic acid analogs, and any combination thereof. Artificial nucleic acid analogs include, for example, glycol nucleic acid, locked nucleic acid, morpholino nucleic acid, peptide nucleic acid, threose nucleic acid, and any combination of such analogs. Nucleic acids should also be understood to include, for example, modified nucleobases and artificial nucleobases.

The terms “nucleic acid” and “nucleic acid sequence” and variations of each term should be understood as used herein to interchangeably refer to a nucleic acid molecule or to a series of letters that indicate the order of nucleotides in a nucleic acid as appropriate in the context of the relevant term.

The term “gene of interest” as used herein should be understood to refer to a nucleotide sequence encoding a gene product of interest. Such a gene product of interest should be understood to refer to a gene product intended to be evolved in a circuitous evolution process as disclosed herein. It will be appreciated that the term “gene of interest” includes variations of the gene of interest that are a result of the circuitous evolution process disclosed herein. A person of ordinary skill will appreciate that a gene of interest could be any nucleic acid encoding a gene product to be evolved.

The terms “protein” and “peptide” and derivatives thereof as used herein should be understood to refer to, for example, molecules including amino acids, and analogs, and derivatives of amino acids.

The term “protein” as used herein should be understood to mean proteins, polypeptides, and peptide of any size, structure, or function. The term as used herein should be understood to refer to a plurality of amino acids linked by peptide bonds. Proteins, as understood herein, include naturally occurring and non-naturally occurring amino acids. Such naturally occurring and non-naturally occurring amino acids may also be modified by addition of one or more chemical entity. Furthermore, proteins may include naturally occurring and non-naturally occurring amino acids analogs and/or derivatives. A person of ordinary skill will understand that proteins may be a single molecule or a plurality of molecules in a complex. Proteins as understood herein include protein fragments, naturally occurring molecules, synthetic molecules, recombination molecules, and any combination of the afore mentioned molecules.

The term “lipid” should be understood as used herein to refer to, for example, diglycerides, fat-soluble vitamins (such as vitamins A, D, E, and K), fatty acids, glycerolipids, glycerophospholipids, monoglycerides, phospholipids, polyketides, prenols, saccharolipids, sphingolipids, sterols, triglycerides, waxes, and analogs and derivatives of the afore mentioned.

The term “carbohydrate” should be understood as used herein to refer to, for example, monosaccharides, disaccharides, oligosaccharides, polysaccharides, and analogs and derivatives of the afore mentioned.

### Nucleic Acid Sequences

One aspect of the invention provides a nucleic acid sequence selected from the group consisting of: a first nucleic acid sequence that includes 5′ to 3′: at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; a second nucleic acid sequence that includes 5′ to 3′: at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence, wherein the 5′ UTR sequence, the one or more non-structural protein(s) coding sequence, the capsid protein coding sequence, the one or more structural protein(s) coding sequence(s), and the 3′ UTR sequence are coding sequences of a Togaviridae virus; and a third nucleic acid sequence that includes 5′ to 3′: at least part of a capsid protein coding sequence, at least part of one or more structural genes coding sequence(s), and at least part of a 3′ UTR sequence, wherein the capsid protein coding sequence, the one or more structural proteins coding sequence(s), and the 3′ UTR sequence are sequences of a Togaviridae virus.

Promoters useful in the present invention include, but are not limited to, a polynucleotide motif related to the recruitment of VP64-p65-Rta elements, miniCMV promoter, a motif ten element and downstream promoter element, a polynucleotide motif encompassing a TATA box, a polynucleotide motif related to the recruitment of p65, a polynucleotide motif related to the recruitment of p65, a polynucleotide motif related to the recruitment of Rta, a polynucleotide motif related to the recruitment of VP64, SP6 promoter in a mammalian expression vector system, T3 promoter in a mammalian expression vector system, T7 promoter in a mammalian expression vector system, a tetracycline response element, and a tetracycline-inducible promoter.

In some embodiments, the Togaviridae virus is an Alphavirus.

In some embodiments, the Alphavirus is Sindbis virus.

In some embodiments, the 5′ UTR sequence is SEQ ID NO. 1.

In some embodiments, the one or more non-structural protein(s) coding sequence(s) is any one or more of SEQ ID No. 2, 3, 4, and 5.

In some embodiments, the gene of interest coding sequence and the sub-genomic promoter coding sequence are operably linked.

In some embodiments, the sub-genomic promoter coding sequence is 26S promoter coding sequence.

In some embodiments, the 26S promoter coding sequence is SEQ ID NO. 6.

In some embodiments, the capsid protein coding sequence is SEQ ID NO. 7.

In some embodiments, each of the first protease cleavage signal coding sequence and the second protease cleavage signal is a self-cleaving peptide coding sequence.

In some embodiments, each of the first protease cleavage signal coding sequence and the second protease cleavage signal is independently selected from the group consisting of equine rhinitis A virus E2A coding sequence, foot-and-mouth disease virus F2A coding sequence, porcine teschovirus-1 2A P2A coding sequence, and those assigned as virus 2A T2A coding sequence.

In some embodiments, the self-cleaving peptide coding sequence is SEQ ID NO. 8.

A person of ordinary skill in the art will appreciate that the first self-cleaving peptide coding sequence and/or the second self-cleaving peptide coding sequence can be substituted with any coding sequence that induces ribosomal skipping.

In some embodiments, the one or more structural protein(s) coding sequence(s) is at least a part of the E3 structural protein coding sequence.

In some embodiments, the E3 structural protein coding sequence is SEQ ID NO. 9.

In some embodiments, the one or more structural protein(s) coding sequence(s) is at least a part of the E2 structural protein gene coding sequence.

In some embodiments, the E2 coding sequence is SEQ ID NO. 10.

In some embodiments, the one or more structural protein(s) coding sequence(s) is at least a part of the 6K structural gene coding sequence.

In some embodiments, the 6K coding sequence is SEQ ID NO. 11.

In some embodiments, the one or more structural protein(s) coding sequence(s) is at least a part of the E1 structural gene coding sequence.

In some embodiments, the E1 coding sequence is SEQ ID NO. 12.

In some embodiments, the 3′ UTR sequence is SEQ ID NO. 13.

In some embodiments, the gene of interest encodes a biomolecule.

In some embodiments, the gene of interest encodes at least one precursor of a biomolecule.

In some embodiments, the gene of interest encodes at least one enzyme of a biosynthetic pathway of a biomolecule.

In some embodiments, the second nucleic acid sequence further includes at least a part of an RNA polynucleotide that encodes a non-coding RNA necessary for viral replication coding sequence.

In some embodiments, the third nucleic acid sequence further includes at least a part of an RNA polynucleotide that encodes a non-coding RNA necessary for viral replication coding sequence.

In some embodiments, the RNA polynucleotide that encodes a non-coding RNA necessary for viral replication coding sequence is hepatitis delta virus (HDV) ribozyme coding sequence.

In some embodiments, the hepatitis delta virus (HDV) ribozyme coding sequence is SEQ ID NO. 17.

In an embodiment, the RNA polynucleotide that encodes an RNA necessary for viral replication coding sequence is any one of SEQ ID NO. 7 to 13.

In an embodiment, the second nucleic acid sequence further includes at least a part of a polyadenylation signal coding sequence.

In an embodiment, the third nucleic acid sequence further includes at least a part of a polyadenylation signal coding sequence.

In some embodiments, the polyadenylation signal coding sequence is a bGH PolyA coding sequence.

In some embodiments, the bGH PolyA coding sequence SEQ ID No. 18.

In some embodiments, the polyadenylation signal coding sequence is any one of SEQ ID NO. 16 and 18.

In some embodiments, the second nucleic acid sequence further includes at least a part of a cis-acting element that promotes nuclear export of an incompletely spliced RNA.

In some embodiments, the third nucleic acid sequence further includes at least a part of a cis-acting element that promotes nuclear export of an incompletely spliced RNA.

In some embodiments, the cis-acting element that promotes nuclear export of an incompletely spliced RNA is a constitutive transport element (CTE).

In some embodiments, the constitutive transport element (CTE) is a type D retrovirus constitutive transport element.

In some embodiments, the constitutive transport element (CTE) is an RNA hairpin motif that binds transporter associated with antigen processing (TAP) host factors.

In some embodiments, the constitutive transport element (CTE) is SEQ ID NO. 14.

In some embodiments, the constitutive transport element (CTE) is SEQ ID NO. 15.

### Method of Stabilising a Recombinant Non-Competent Alphavirus Genome Adjacent a 3′ End

Another aspect of the invention provides a method of stabilising a recombinant non-competent genome adjacent a 3′ end, the method includes inserting a gene of interest in frame with an open reading frame of at least a part of at least one structural protein coding sequence in a non-competent viral vector, wherein the at least one structural protein coding sequence is a Togaviridae virus structural protein coding sequence. It will be appreciated that a method of stabilising a recombinant non-competent genome adjacent a 3′ end, the method including inserting a gene of interest in frame with an open reading frame of at least a part of at least one non-structural protein coding sequence in a non-competent viral vector, wherein the at least one non-structural protein coding sequence is a Togaviridae virus non-structural protein coding sequence is also contemplated.

As used herein, the term “viral vector” should be understood to refer to a nucleic acid that includes a viral genome which, when introduced into a suitable host cell or used as a template for in vitro transcription, can produce viral RNA which can be replicated and packaged into viral particles. Such viral particles transfer the viral genome into another host cell. It will be appreciated that the term “viral vector” extends to at least part of a viral genome, i.e., truncated and/or partial viral genomes. In some embodiments, a viral vector is provided that lacks one or more gene encoding a protein essential for generation of an infectious viral particle. As used herein the term “viral particle” should be understood to refer to viral nucleic acid(s) that encode one or more viral coat protein(s) and/or viral lipid envelope. As used herein, the term “infectious viral particle” as herein should be understood to refer to a viral particle that can transport at least part of a viral nucleic acid into a suitable host cell.

A person skilled in the art will appreciate, as an example, that the viral vector can be linearized via a restriction digest and used as a template for in vitro transcription if the viral coding sequence is downstream of an SP6 or T7 promoter using ordinary kits (i.e., mMESSAGE mMACHINE™ SP6 Transcription Kit, Thermo Fisher, #AM1340).

In some embodiments, a non-competent viral vector is provided that lacks one or more gene(s) encoding at least one protein essential for generation of an infectious viral particle.

As used herein the term “viral particle” should be understood to refer to viral nucleic acid(s) that encode one or more viral coat protein(s) and/or viral lipid envelope. As used herein, the term “infectious viral particle” as herein should be understood to refer to a viral particle that can transport at least part of a viral nucleic acid into a suitable host cell.

In some embodiments, the Togaviridae virus structural protein coding sequence is selected from the group consisting of the capsid coding sequence, the E3 coding sequence, the E2 coding sequence, the 6K coding sequence, and the E1 coding sequence of a Togaviridae virus.

In some embodiments, the capsid coding sequence is SEQ ID NO. 7.

In some embodiments, the one or more structural protein(s) coding sequence(s) is at least a part of the E3 structural protein coding sequence.

In some embodiments, the E3 structural protein coding sequence is SEQ ID NO. 9.

In some embodiments, the one or more structural protein(s) coding sequence(s) is at least a part of the E2 structural protein gene coding sequence.

In some embodiments, the E2 coding sequence is SEQ ID NO. 10.

In some embodiments, the one or more structural protein(s) coding sequence(s) is at least a part of the 6K structural gene coding sequence.

In some embodiments, the 6K coding sequence is SEQ ID NO. 11.

In some embodiments, the one or more structural protein(s) coding sequence(s) is at least a part of the E1 structural gene coding sequence.

In some embodiments, the E1 coding sequence is SEQ ID NO. 12.

The disclosed method of stabilising a recombinant non-competent genome adjacent a 3′ end positively selects for an accumulation of mutations that increase solubility of a protein encoded by the gene of interest and/or host codon optimization for the gene of interest. Conversely, the disclosed method of stabilising a recombinant non-competent genome adjacent a 3′ end negatively selects against deleterious mutations within the gene of interest, such as nonsense mutations and/or missense mutations that decrease solubility of a protein encoded by the gene of interest.

The disclosed method of stabilising a recombinant non-competent genome adjacent a 3′ end decreases frequency of a nonsense mutation in the gene of interest relative to wild-type gene of interest.

The term “wild-type” as used herein with respect to a gene should be understood to refer to a gene when the gene is found in its natural, non-mutated, i.e., unchanged, form.

In some embodiments, the method of stabilising a recombinant non-competent genome adjacent a 3′ end decreases frequency of a nonsense mutation in the gene of interest at least 2 orders of magnitude relative to wild-type gene of interest.

In some embodiments, the method of stabilising a recombinant non-competent genome adjacent a 3′ end decreases frequency of a nonsense mutation in the gene of interest at least 4 orders of magnitude relative to wild-type gene of interest.

In some embodiments, the method of stabilising a recombinant non-competent genome adjacent a 3′ end decreases frequency of a nonsense mutation in the gene of interest at least 8 orders of magnitude relative to wild-type gene of interest.

In some embodiments, the method of stabilising a recombinant non-competent genome adjacent a 3′ end decreases frequency of a nonsense mutation in the gene of interest at least 16 orders of magnitude relative to wild-type gene of interest.

In some embodiments, the method of stabilising a recombinant non-competent genome adjacent a 3′ end decreases frequency of a nonsense mutation in the gene of interest at least 32 orders of magnitude relative to wild-type gene of interest.

In some embodiments, the method of stabilising a recombinant non-competent genome adjacent a 3′ end decreases frequency of a nonsense mutation in the gene of interest at least 64 orders of magnitude relative to wild-type gene of interest.

In some embodiments, the method of stabilising a recombinant non-competent genome adjacent a 3′ end decreases frequency of a nonsense mutation in the gene of interest at least 100 orders of magnitude relative to wild-type gene of interest.

In some embodiments, the method of stabilising a recombinant non-competent genome adjacent a 3′ end decreases frequency of a nonsense mutation in the gene of interest at least 120 orders of magnitude relative to wild-type gene of interest.

An example of a method for stabilising a recombinant non-competent genome adjacent a 3′ end includes the insertion of a disrupted blasticidin resistance gene (C56X) in frame with the Sindbis viral structural glycoproteins via scarless cloning approaches. Primers containing a 5′ extension with appropriate homology arms to either the upstream capsid sequence, or the downstream protease cleavage signal were designed to amplify and then mediate insertion of the disrupted blasticidin resistance gene fragment (C56X) in-frame with the viral open reading frame. Such methods can include Gibson assembly, gene synthesis, or Golden Gate and would follow ordinary molecular cloning, screening and validation procedures that would include Sanger sequencing to check that the disrupted blasticidin resistance gene (C56X) fragment was inserted correctly in frame.

The method of stabilising a recombinant non-competent genome adjacent a 3′ end increases frequency of a synonymous mutation in the gene of interest relative to wild-type gene of interest. The term “synonymous mutation”, also known as “silent substitution” or “silent mutation”, should be understood herein to refer to a mutation that does not result in a change in the produced amino acid, i.e., a consequence of the degeneracy of the genetic code. A person of ordinary skills will understand that a synonymous mutation, although coding for the same produced amino acid, may affect transcription, may produce alternative splice variants of the messenger RNA, and consequently, translation of a resultant protein. A person of ordinary skill will understand that such a difference in translation may result in a protein having different characteristics, including differences in protein expression, primary structure, secondary structure, tertiary structure, quaternary structure, emulsification, glass transition temperature optimum, isoelectric point, melting point, molecular weight, pH optimum, solubility, and/or surface hydrophobicity.

In some embodiments, the method of stabilising a recombinant non-competent genome adjacent a 3′ end increases frequency of a synonymous mutation in the gene of interest by at least 2 orders of magnitude relative to wild-type gene of interest.

In some embodiments, the method of stabilising a recombinant non-competent genome adjacent a 3′ end increases frequency of a synonymous mutation in the gene of interest by at least 4 orders of magnitude relative to wild-type gene of interest.

In some embodiments, the method of stabilising a recombinant non-competent genome adjacent a 3′ end increases frequency of a synonymous mutation in the gene of interest by at least 8 orders of magnitude relative to wild-type gene of interest.

In some embodiments, the method of stabilising a recombinant non-competent genome adjacent a 3′ end increases frequency of a synonymous mutation in the gene of interest by at least 16 orders of magnitude relative to wild-type gene of interest.

In some embodiments, the method of stabilising a recombinant non-competent genome adjacent a 3′ end increases frequency of a synonymous mutation in the gene of interest by at least 32 orders of magnitude relative to wild-type gene of interest.

In some embodiments, the method of stabilising a recombinant non-competent genome adjacent a 3′ end increases frequency of a synonymous mutation in the gene of interest by at least 40 orders of magnitude relative to wild-type gene of interest.

An example of a method for stabilising a recombinant non-competent genome adjacent a 3′ end includes insertion of the SP6 RNA polymerase (SP6 RNAP) coding sequence as a gene of interest in frame with the partial capsid and structural glycoprotein coding sequences. SP6 RNAP is typically expressed in a bacterial context and would be expected to express relatively poorly in mammalian BHK-21 cells. Using standard scarless cloning techniques such as Gibson Assembly, gene synthesis, or Golden Gate cloning, along with validation techniques such as Sanger sequencing to check for the in-frame insertion of the SP6 RNAP coding sequence, serial passaging of SP6 RNAP enriched for mutations that would improve protein expression and solubility due to the carryon effect of poor protein expression to the downstream structural glycoproteins of the viral particle.

### Methods of Increasing Viral Titre

Another aspect of the invention provides a method of increasing viral titre selected from the group consisting of a first method of increasing viral titre, the first method including: providing a suitable host cell; introducing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are coding sequences of a Togaviridae virus, into the suitable host cell; introducing one or more first complement nucleic acid sequence including 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence, wherein the 5′ UTR sequence, the one or more non-structural protein(s) coding sequence, the capsid protein coding sequence, the one or more structural genes coding sequence(s), and the 3′ UTR sequence are sequences of a Togaviridae virus into the suitable host cell; enabling expression of the viral vector in the suitable host cell; and enabling expression of the nucleic acid sequence in the suitable host cell; and a second method of increasing viral titre, the second method including: providing a suitable host cell; introducing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, and the E1 coding sequence, and the 3′ UTR sequence are sequences of a Togaviridae virus, into the suitable host cell; introducing one or more second complement nucleic acid sequence including 5′ to 3′ at least part of a capsid protein coding sequence, at least part of one or more structural genes coding sequence(s), and at least part of a 3′ UTR sequence, wherein the capsid protein coding sequence, the one or more structural genes coding sequence(s), and the 3′ UTR sequence coding sequences of a Togaviridae virus into the suitable host cell; enabling expression of the viral vector in the suitable host cell; and enabling expression of the nucleic acid sequence in the suitable host cell. It will be appreciated that the order of introduction and expression of the viral vector and the nucleic acid to the suitable host cell may occur in any order or simultaneously.

The term “viral titre” as used herein should be under to refer to the number of viral particles as measured by quantitative polymerase chain reaction (qPCR), or by performing a plaque assay in which viral particles of an unknown quantity are applied to a suitable host cell monolayer under a low viscosity overlay media, or by transducing a suitable host cell monolayer with viruses encoding a reporter gene and performing a count of the number of host cells within that monolayer that expresses the reporter gene, as determined by serial dilution of the viral particles.

The term “host cell,” as used herein, should be understood to refer to a cell that can host a viral vector and/or a virus. A cell can host a viral vector if it supports expression of genes of a viral vector, replication of a viral genome, and/or the generation of viral particles. One criterion to determine whether a cell is a suitable host cell for a given viral vector is to determine whether the cell can support the viral life cycle of a wild-type viral genome that the viral vector is derived from.

A preferred host cell useful for purposes of the present invention includes a host cell can support the life cycle of a member of the Togaviridae family. A preferred host cell useful for purposes of the present invention includes a host cell can support the life cycle of a member of the Alphavirus genus. A particularly preferred host cell useful for purposes of the present invention includes a host cell can support the life cycle of Sindbis virus. Preferably, the host cell includes and is capable of expression of a heparan sulfate nucleic acid sequence. Also preferably, the host cell includes and is capable of expression of a laminin receptor nucleic acid sequence. Also preferably, the host cell is a HEK293 cell. Also preferably, the host cell is a chicken embryo cell line cell. Also preferably, the host cell is an Aedes albopictus cell. Also preferably, the Aedes albopictus cell is a U4.4 cell. Also preferably, the Aedes albopictus cell is a C6/36 cell. Also preferably, the Aedes albopictus cell is a C7-10 cell. Also preferably, the host cell is a suitable mammalian cell. Also preferably, the host cell is a BHK-21 cell.

An inducible circuit can be constructed by integrating an intact or disrupted T7 RNA polymerase gene into a suitable host cell. Transfection of a complement plasmid bearing a T7 promoter driving the expression of the viral complement required for viral maturation and egress can therefore link the function of the gene-of-interest to that of the virus by require activation of the T7 promoter as a proxy. For example, in a hypothetical scenario, a viral particle expressing a gene of interest DNA-editing agent such as a base editor phenotypically links the accumulation of gain-of-function mutations in the gene of interest with the reversion of the stop codon occurring in the disrupted T7 ORF. In this case, mutations that are beneficial to the gene of interest DNA editing agent such as a base editor increase the efficiency at which the stop codon is corrected, and therefore establishes a conditional, selective benefit for evolved gene-of-interest variants.

A person of ordinary skill will appreciate that increasing viral titre can be performed using a combination of the first method of method of increasing viral titre and second method of method of increasing viral titre.

In some embodiments, the Togaviridae virus is an Alphavirus.

In some embodiments, the Alphavirus is Sindbis virus.

In some embodiments, the 5′ UTR sequence is SEQ ID NO. 1.

In some embodiments, the Togaviridae virus structural protein coding sequence is selected from the group consisting of the capsid coding sequence, the E3 coding sequence, the E2 coding sequence, the 6K coding sequence, and the E1 coding sequence of a Togaviridae virus.

In some embodiments, the capsid coding sequence is SEQ ID NO. 7.

In some embodiments, the one or more structural protein(s) coding sequence(s) is at least a part of the E3 structural protein coding sequence.

In some embodiments, the E3 structural protein coding sequence is SEQ ID NO. 9.

In some embodiments, the one or more structural protein(s) coding sequence(s) is at least a part of the E2 structural protein gene coding sequence.

In some embodiments, the E2 coding sequence is SEQ ID NO. 10.

In some embodiments, the one or more structural protein(s) coding sequence(s) is at least a part of the 6K structural gene coding sequence.

In some embodiments, the 6K coding sequence is SEQ ID NO. 11.

In some embodiments, the one or more structural protein(s) coding sequence(s) is at least a part of the E1 structural gene coding sequence.

In some embodiments, the E1 coding sequence is SEQ ID NO. 12.

In some embodiments, the 3′ UTR sequence is SEQ ID NO. 13.

In some embodiments, the complement nucleic acid sequence further includes at least a part of an RNA polynucleotide that encodes a non-coding RNA necessary for viral replication coding sequence.

In some embodiments, the RNA polynucleotide that encodes a non-coding RNA necessary for viral replication coding sequence is hepatitis delta virus (HDV) ribozyme coding sequence.

In some embodiments, the RNA polynucleotide that encodes an RNA necessary for viral replication coding sequence is any one of SEQ ID NO. 7 to 13.

In some embodiments, the complement nucleic acid sequence further includes at least a part of a polyadenylation signal coding sequence.

In some embodiments, the polyadenylation signal coding sequence is bGH PolyA coding sequence.

In some embodiments, the polyadenylation signal coding sequence is any one of SEQ ID NO. 16 and 18.

In some embodiments, the one or more first complement nucleic acid sequence and the one or more second complement nucleic acid sequence further include at least a part of a cis-acting element that promotes nuclear export of an incompletely spliced RNA.

In some embodiments, the cis-acting element that promotes nuclear export of an incompletely spliced RNA is a constitutive transport element (CTE).

In some embodiments, the constitutive transport element (CTE) is a type D retrovirus constitutive transport element.

In some embodiments, the constitutive transport element (CTE) is an RNA hairpin motif that binds transporter associated with antigen processing (TAP) host factors.

An increase in viral titre can be measured using viral transduction or qPCR of the gene of interest as will be understood by a person of ordinary skill in the art. Such increase in viral titre can occur under drift or non-drift conditions as will be understood by a person skilled in the art.

As used here, the term “drift conditions” refers to genetic drift conditions in which the recombinant, non-competent viral particle containing the gene of interest is serially passaged under permissive conditions such that the viral complement necessary for viral propagation is provided constitutively and unconditionally. This enables the accumulation of mutations that can be either dependent or independent of the function of the gene of interest as the viral particles and its gene products thereof are not operating under selection. As an example, genetic drift conditions can be used to avoid population bottlenecks and the subsequent loss of genetic diversity in the gene of interest, and specifically, for RNA viruses, avoid the loss of memory genomes in a quasi-species distribution. Likewise, a person skilled in the art will also appreciate that ‘drift’ conditions can occur by overexpressing the complement under a strong promoter such as CMV or other RNA pol II promoters or involve the transient transfection of purified RNA expressing the viral complement. Conversely, as used herein, the term “non-drift conditions” refers to serial passaging of the viral particle under selective conditions that enriches for gene of interest or viral variants that have improved fitness in terms of the Darwinian fitness landscape.

In an embodiment, increasing the viral titre using the first method of increasing viral titre or the second method of increasing viral titre increases the viral titre by at least 1.5 orders of magnitude relative to serial passaging conditions in which the second or third nucleic acid (first or second complement nucleic acid) has not been provided transiently or constitutively in a suitable host cell.

In an embodiment, increasing the viral titre using the first method of increasing viral titre or the second method of increasing viral titre increases the viral titre by at least 2 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 3 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 4 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 5 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 6 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 7 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 8 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 9 orders of magnitude relative to serial passaging conditions in which the first or second complement nucleic acid, i.e., the second or third nucleic acid described hereinabove, respectively has not been provided transiently or constitutively in a suitable host cell.

In an embodiment, increasing the viral titre using the first method of increasing viral titre or the second method of increasing viral titre increases the viral titre by at least 10 orders of magnitude relative to serial passaging conditions in which the first or second complement nucleic acid, i.e., the second or third nucleic acid described hereinabove, respectively has not been provided transiently or constitutively in a suitable host cell.

A person skilled in the art will appreciate that, as an example, insertion of an enhanced green fluorescent protein (EGFP) coding sequence in frame with the recombinant, non-competent viral particle, wherein the intact open reading frame occurs downstream of a partial capsid coding sequence and upstream from other structural proteins can produce a robust EGFP fluorescent signal. During serial passaging, a measure of the transducing units/ml (TU/mL) showed that viral titres were as high as 5E+05 TU/mL in the presence of the transfected RNA complement expressing the capsid gene product, with loss of EGFP fluorescence observed when the virus was passaged in the absence of the viral complement.

### Method of Function-Dependent Propagation of a Togaviridae Virus

Another aspect of the invention provides a method of function-dependent propagation of a Togaviridae virus in a suitable host cell, the method including: providing a viral vector including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; linking expression of the at least part of an in-frame Togaviridae virus capsid nucleic acid sequence and expression of the at least part of one or more in-frame Togaviridae virus structural protein(s) encoding nucleic acid sequence(s) to a function of a gene product of the gene of interest,

As used herein, the term “in trans” refers to segmentation of a viral genome such that part of the viral genome may be partitioned either as a separate polynucleotide on a different vector or on a template. The term “template” as used herein should be understood to refer to a nucleic acid, for example an oligonucleotide or polynucleotide, including at least one codon sequence suitable for a template-mediated chemical synthesis. A person skilled in the art will appreciate that the template may, for example, include a plurality of codon sequences, an amplification means, for example, a PCR primer binding site or a sequence complementary thereto, a reactive unit, and any combination of the afore mentioned. Such a reactive unit may for example include a building block, a molecular scaffold, a monomer component, a monomer, another reactant useful in template mediated chemical synthesis, and any combination of the afore mentioned.

In some embodiments, the Togaviridae virus is an Alphavirus.

In some embodiments, the Alphavirus is Sindbis virus.

In some embodiments, the 5′ UTR sequence is SEQ ID NO. 1.

In some embodiments, the Togaviridae virus structural protein coding sequence is selected from the group consisting of the capsid coding sequence, the E3 coding sequence, the E2 coding sequence, the 6K coding sequence, and the E1 coding sequence of a Togaviridae virus.

In some embodiments, the capsid coding sequence is SEQ ID NO. 7.

In some embodiments, the one or more structural protein(s) coding sequence(s) is at least a part of the E3 structural protein coding sequence.

In some embodiments, the E3 structural protein coding sequence is SEQ ID NO. 9.

In some embodiments, the one or more structural protein(s) coding sequence(s) is at least a part of the E2 structural protein gene coding sequence.

In some embodiments, the E2 coding sequence is SEQ ID NO. 10.

In some embodiments, the one or more structural protein(s) coding sequence(s) is at least a part of the 6K structural gene coding sequence.

In some embodiments, the 6K coding sequence is SEQ ID NO. 11.

In some embodiments, the one or more structural protein(s) coding sequence(s) is at least a part of the E1 structural gene coding sequence.

In some embodiments, the E1 coding sequence is SEQ ID NO. 12.

In some embodiments, the 3′ UTR sequence is SEQ ID NO. 13.

An embodiment could include the insertion of a tetracycline-transactivator (tTA) coding sequence as a gene of interest in frame with the partial capsid and structural glycoprotein coding sequences. Serial passaging conditions may include the use of inhibiting concentrations of doxycycline (DOX) between 0 ng/ml to 1000 ng/mL that would otherwise repress the function of tTA. A mammalian expression plasmid bearing a tTA-responsive TETO7 promoter and the downstream viral complement components necessary for viral maturation and egress could be transiently transfected into naïve BHK-21 cells. Transduction of transfected BHK-21 cells mediate the interaction between the tTA gene of interest in the viral particle and the activation of the TETO7 promoter leading to expression of the viral complement. Addition of 1 ng/ml to 1000 ng/ml of DOX would inhibit activation of the TETO7 promoter and prevent viral maturation. Therefore, function-dependent propagation occurs based on the accumulation of mutations that confer a DOX-insensitive phenotype to tTA, which can subsequently be isolated and sequenced using standard techniques that can include Sanger sequencing or nanopore flow cells.

### Method of Evolution of a Gene Product of a Gene of Interest

Another aspect of the invention provides a method of evolution of a gene product of a gene of interest, the method including: providing one or more viral vector(s) comprising 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; providing one or more first complement nucleic acid sequence(s) comprising 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence into the population of suitable host cells, wherein the 5′ UTR sequence, the one or more non-structural protein(s) coding sequence, the capsid protein coding sequence, the one or more structural genes coding sequence(s), and the 3′ UTR sequence are sequences of a Togaviridae virus, and wherein expression of the one or more first complement nucleic acid sequence(s) is linked to a function of a gene product of the gene of interest; introducing the one or more viral vector(s) to a population of suitable host cells; introducing the one or more first complement nucleic acid sequence(s) to the population of suitable host cells; allowing maturation and egress of one or more mature virus(es) that comprise the gene of interest or a variant thereof from the population of suitable host cells; recovering the one or more mature virus(es); introducing the one or more mature virus(es) and one or more first complement nucleic acid sequence(s) into a population of naïve suitable host cells; allowing further maturation and egress of further one or more mature virus(es) that comprise the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s).

Another aspect of the invention provides a method of evolution of a gene product of a gene of interest selected from the group consisting of: a first method of evolution of a gene product of a gene of interest, the first method including: providing one or more viral vector(s) including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus; providing one or more first complement nucleic acid sequence(s) including 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence into the population of suitable host cells, wherein the 5′ UTR sequence, the one or more non-structural protein(s) coding sequence, the capsid protein coding sequence, the one or more structural genes coding sequence(s), and the 3′ UTR sequence are sequences of a Togaviridae virus, and wherein expression of the one or more first complement nucleic acid sequence(s) is linked to a function of a gene product of the gene of interest; introducing the one or more viral vector(s) to a population of suitable host cells; introducing the one or more first complement nucleic acid sequence(s) to the population of suitable host cells; allowing maturation and egress of one or more mature virus(es) that include the gene of interest or a variant thereof from the population of suitable host cells; recovering the one or more mature virus(es); introducing the one or more mature virus(es) and one or more first complement nucleic acid sequence(s) into a population of naïve suitable host cells; allowing further maturation and egress of further one or more mature virus(es) that include the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s); and a second method of evolution of a gene product of a gene of interest, the second method including: providing one or more viral vector(s) including 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, and the E1 coding sequence, and the 3′ UTR sequence are sequences of a Togaviridae virus; providing one or more second complement nucleic acid sequence(s) including 5′ to 3′ at least part of a capsid protein coding sequence, at least part of one or more structural genes coding sequence(s), and at least part of a 3′ UTR sequence, wherein the capsid protein coding sequence, and the one or more structural genes coding sequence(s), and the 3′ UTR sequence are sequences of a Togaviridae virus, and wherein expression of the one or more second complement nucleic acid sequence(s) is linked to a function of a gene product of the gene of interest; introducing the one or more viral vector(s) to a population of suitable host cells; introducing the one or more complement nucleic acid(s) to the population of suitable host cells; allowing maturation and egress of one or more mature virus(es) that include the gene of interest or a variant thereof from the population of suitable host cells; recovering the one or more mature virus(es); introducing the one or more mature virus(es) and one or more second complement nucleic acid sequence(s) into a population of naïve suitable host cells; allowing further maturation and egress of further one or more mature virus(es) that include the gene of interest or a variant thereof from the population of suitable host cells; recovering the further one or more mature virus(es); isolating one or more nucleic acid sequence(s) from the further one or more mature virus(es) to provide one or more isolated nucleic acid sequence(s); and isolating the gene of interest or a variant thereof from the one or more isolated nucleic acid sequence(s). It will be appreciated that the steps may be repeated to further evolve the gene product of the gene of interest. It will also be appreciated that the order of introducing the one or more viral vector(s) to a population of suitable host cells and introducing the one or more first and/or second complement nucleic acid(s) to a population of suitable host cells may occur in any order or simultaneously.

A person of ordinary skill will appreciate that a function of a gene product of a gene of interest that favours expression of the Togaviridae virus capsid gene will favour viral maturation and egress of a mature virus from the suitable host cell. A person of ordinary skill will also appreciate that a function of the gene product of the gene of interest that does not favour expression of the Togaviridae virus capsid gene will either not favour or less efficiently favour viral maturation and egress of a mature virus from the suitable host cell as compared to a function of the gene product of the gene of interest that favours expression of the Togaviridae virus capsid gene. In effect, favouring of expression of the Togaviridae virus capsid gene will concomitantly favour expression of the gene of interest.

A person of ordinary skill will appreciate that stringency of a method of evolving a gene product of a gene of interest can be modulated. The first method of evolution of a gene product of a gene of interest disclosed herein may be considered as a “relaxed” method of evolving a gene product of a gene of interest. On the other hand, the second method of evolution of a gene product of a gene of interest disclosed herein may be considered as a “stringent” method of evolving a gene product of a gene of interest.

A person of ordinary skill will appreciate that evolution of a gene product of the gene of interest can be performed using any combination of the first method of evolution of a gene product of a gene of interest and second method of evolution of a gene product of a gene of interest.

In some embodiments, the Togaviridae virus is an Alphavirus.

In some embodiments, the Alphavirus is Sindbis virus.

In some embodiments, the 5′ UTR sequence is SEQ ID NO. 1.

In some embodiments, the Togaviridae virus structural protein coding sequence is selected from the group consisting of the capsid coding sequence, the E3 coding sequence, the E2 coding sequence, the 6K coding sequence, and the E1 coding sequence of a Togaviridae virus.

In some embodiments, the capsid coding sequence is SEQ ID NO. 7.

In some embodiments, the one or more structural protein(s) coding sequence(s) is at least a part of the E3 structural protein coding sequence.

In some embodiments, the E3 structural protein coding sequence is SEQ ID NO. 9.

In some embodiments, the one or more structural protein(s) coding sequence(s) is at least a part of the E2 structural protein gene coding sequence.

In some embodiments, the E2 coding sequence is SEQ ID NO. 10.

In some embodiments, the one or more structural protein(s) coding sequence(s) is at least a part of the 6K structural gene coding sequence.

In some embodiments, the 6K coding sequence is SEQ ID NO. 11.

In some embodiments, the one or more structural protein(s) coding sequence(s) is at least a part of the E1 structural gene coding sequence.

In some embodiments, the E1 coding sequence is SEQ ID NO. 12.

In some embodiments, the 3′ UTR sequence is SEQ ID NO. 13.

In some embodiments, the one or more first complement nucleic acid sequence(s) further include(s) at least a part of an RNA polynucleotide that encodes a non-coding RNA necessary for viral replication coding sequence.

In some embodiments, the one or more second complement nucleic acid sequence(s) further include(s) at least a part of an RNA polynucleotide that encodes a non-coding RNA necessary for viral replication coding sequence.

In some embodiments, the RNA polynucleotide that encodes a non-coding RNA necessary for viral replication coding sequence is hepatitis delta virus (HDV) ribozyme coding sequence.

In some embodiments, the RNA polynucleotide that encodes a non-coding RNA necessary for viral replication coding sequence is any one of SEQ ID NO. 7 to 13.

In some embodiments, the complement nucleic acid sequence further includes at least a part of a polyadenylation signal coding sequence.

In some embodiments, the polyadenylation signal coding sequence is bGH PolyA coding sequence.

In some embodiments, the polyadenylation signal coding sequence is any one of SEQ ID NO. 16 and 18.

In some embodiments, the one or more first complement nucleic acid sequence and the one or more second complement nucleic acid sequence further include at least a part of a cis-acting element that promotes nuclear export of an incompletely spliced RNA

In some embodiments, the cis-acting element that promotes nuclear export of an incompletely spliced RNA is a constitutive transport element (CTE).

In some embodiments, the constitutive transport element (CTE) is a type D retrovirus constitutive transport element.

In some embodiments, the constitutive transport element (CTE) is an RNA hairpin motif that binds transporter associated with antigen processing (TAP) host factors.

An embodiment includes use of a transgenic cell line such as a modified BHK-21 cell line to express lentivirally integrated T7 or SP6 RNA polymerase (RNAP). T7 or SP6 RNAP is typically used to express transcripts under in vitro conditions as they have a clearly defined transcription start site (TSS). Therefore, insertion of T7 or SP6 RNAP into a cell line could allow the expression of the ‘relaxed’ viral complement which contains upstream non-coding and coding elements in addition to the capsid protein coding sequence. Expression of the ‘relaxed’ complement requires an expression system capable of transcribing long, non-coding RNAs (IncRNAs) that do not undergo splicing. Therefore, one can appreciate that a ‘relaxed’ circuit would be conceived around modulating the expression of the transgenic T7 or SP6 RNAP cell line, when a complementary plasmid bearing the respective T7 or SP6 promoter and the downstream viral components are introduced into this transgenic cell line. Conversely, a stringent circuit would involve the construction of a cell line expressing tetracycline-transactivator (tTA), which is an inducible RNA pol II-type promoter. As the ‘stringent’ circuit does not contain upstream non-coding and coding elements beyond the Kozak sequence of the capsid gene fragment, the variant of the promoter is linked to those that would mediate nuclear export and translation. A mammalian expression plasmid bearing a tTA-interacting promoter such as TETO7 and the downstream capsid coding sequence can be introduced into a transgenic cell line expressing intact or non-intact tTa, wherein the function of the gene of interest would be dependent on activating the function of tTa. One can appreciate that an example application of this would be to use a gene of interest, such as a DNA-editing agent that can correct a stop codon in either a disrupted T7 or SP6 RNAP open reading frame, or a disrupted tTa open reading frame and switch between “relaxed” and “stringent” circuits as a function of measured viral titre after each round of serial passaging.

While the present invention has been described with reference to Sindbis virus, other Togaviridae viruses, e.g., Chikungunya virus, Semliki Forest virus, Ross River virus, o′nyong-nyong virus, Mayaro virus, Eastern equine encephalitis virus (EEEV), Venezuelan equine encephalitis virus (VEEV), and Western equine encephalitis virus (WEEV) are contemplated as being useful viruses for use in the present invention.

Further, some aspects of this invention provide kits including reagents, vectors, cells, software, systems, and/or apparatuses for carrying out the methods provided herein. For example, in some embodiments, a kit for performing evolution of a gene product of a gene of interest that includes one or more of the nucleic acid sequence(s) disclosed herein, one or more complement nucleic acid sequence(s) disclosed herein, one or more host cell(s) disclosed herein. Typically, the kit will also include instructions for performing the evolution of the gene product of the gene of interest.

The present invention is described further below in examples which are intended to describe the invention without limiting the scope thereof.

In the examples below, the following materials and methods were used.

### EXAMPLES

### General Cell Culture

BHK-21 [C-13] cells (#CCL-10) (hereinafter BHK-21) were grown in a humidified 37° C. (5% CO₂) incubator in MEM α (ThermoFisher, #32571101) supplemented with 1% Penicillin-Streptomycin (% v/v), 5% Fetal Bovine Serum (FBS) (ThermoFisher Scientific, #16000044) (% v/v) and 10% tryptose phosphate broth (TPB) (ThermoFisher, #CM0283B) (% v/v), referred to as BHK-21 Full Medium. During transduction, cells were maintained in TM diluent as described “Current Protocols in Microbiology”, Coico (2005).

### Domain Mutagenesis

The GeneMorph II EZClone Domain Mutagenesis kit was used, as per manufacturer's instructions, to facilitate random mutagenesis of a gene of interest SP6 prior to mRNA synthesis for serial passaging. Briefly, gene-specific primers (SEQ ID NO. 20 and 21) were used to amplify the gene of interest; input DNA amounts varied from between 10 ng to 1 ug to capture low, medium, and high mutagenesis rates. Each reaction was performed in duplicate, and a general annealing temperature of 60° C. was used. For the EZClone reaction, thermocycling conditions were as described, except that an extension parameter of 3 minutes/kb length of the plasmid was used. Next, up to 15 to 20 transformations using chemically competent E. coli (NEB, #C3040H) were performed as per manufacturer's instructions and allowed to incubate at 37° C. overnight. Colonies were harvested using a cell scraper (Sigma Aldrich, #CLS3011-100EA) and a Midiprep kit (QIAGEN, #12145).

### mRNA Synthesis

Plasmids (SEQ ID NO. 19, 31, 40, 41) (3 μg) were linearized prior to mRNA synthesis by using XbaI restriction enzyme. Phenol-chloroform extraction following manufacturer's protocol (Thermofisher, #15593031) was used to purify linearized plasmid, and 500 ng of input DNA was used to produce mRNA, with LiCl purification steps taken immediately after, using the mMESSAGE mMACHINE™ SP6 Transcription Kit (ThermoFisher, #AM1340), as outlined English, J. G. et al. Cell 178, 748-761.e17 (2019). Next, mRNA was quantified via nanodrop spectrophotometer. mRNA integrity was assessed by gel electrophoresis.

### Packaging of SINV Particles (Referred to as Round 0 (R0))—mRNA Electroporation

1×10⁶ BHK-21 cells were nucleofected using a total of 20 μg of mRNA (1:1 of pSinCapsid (SEQ ID NO. 27), SIN2A-Envelope-EGFP (SEQ ID NO. 19), SIN2A-SP6-Envelope (SEQ ID NO. 31), or SIN2A-BSR-Envelope (SEQ ID NO. 40), or SIN2A-BSR (C56X)-Envelope (SEQ ID NO. 41)) using the Amaxa SE Cell Line 4D-Nucleofector™ X Kit L (Lonza, #V4XC-1024) with the 4D-Nucleofector™ System (Lonza) on the C-034 program. Nucleofected cells were plated in BHK-21 Full Medium and were incubated as described above under “General Cell Culture” for 20-24 hours. Supernatant was harvested and centrifuged at 500 g for 5 minutes at room temperature and filtered using a 0.45 μm filter (Merck Millipore #SLHV033RS) and a sterile 10 mL Luer lock syringe (MicroAnalytix, #MS S3P10LL). Viral supernatant was stored at 4° C. for up to 1 month.

### Determining Optimum Transfection Time Point for RNA Transfection of Split Viral Vectors

1×10⁵ BHK-21 cells were plated in a 6 well plate (Corning Costar, #CLS3516-50EA) and incubated as described under “General Cell Culture” for up to 24 hours. Briefly, 1 mL of virus containing supernatant as prepared in “Packaging of SINV Particles (referred to as Round 0 (R0)—mRNA Electroporation”, were used to transduce plated BHK-21 cells by incubating the virus containing supernatant on the cell monolayer for 1 hour with intermittent, gentle shaking every 15 minutes under “General Cell Culture” conditions. Next, 20 minutes prior to the aspiration of the virus containing supernatant from the cell monolayer, 1 μg of in vitro transcribed and purified SINV biosensor RNA (SEQ ID NO. 47), as previously prepared under “mRNA synthesis”, was mixed with 4 μL of Lipofectamine 2000 (ThermoFisher, # 11668030) in 500 μL Opti-MEM Reduced Serum Medium (GlutaMAX Supplement) (ThermoFisher, #51985034) and incubated for 20 minutes at room temperature, referred to hereafter as the “transfection mixture”. Next, virus containing supernatant was aspirated from the cell monolayer, followed by 2×DPBS washing (ThermoFisher #14190250). The transfection mixture was immediately applied to the transduced cell monolayer. After 4 hours, the transfection mixture was removed, followed by 2×DPBS washing, and the cell culture well was replaced with BHK-21 Full Media and returned to “General Cell Culture” conditions.

### Viral Transduction for Serial Passaging

Briefly, a single round of a campaign includes four technical replicates run in parallel. Naïve BHK-21 cells were seeded in T25 flasks at 2.5×10⁵ cells/flask and incubated for 24 hours. Subsequently, each T25 flask was transfected using 5 μg of complement plasmid (SEQ ID NO. 28) and 15 μL of TransIT-2020 Transfection Reagent (Mirus Bio, #MIR5400) made up to a total volume of 500 μL with Opti-MEM Reduced Serum Medium (GlutaMAX Supplement) (ThermoFisher, #51985034) according to the manufacturer's recommendations. After 6 hours, transfection media was removed and 1 mL of viral supernatant or viral supernatant diluted in TM diluent was prepared as described “Current Protocols in Microbiology”, Coico (2005).

Next, 1 mL of diluted (in TM diluent) or undiluted viral supernatant was applied to transfected BHK-21 cells with intermittent shaking every 15 minutes for a 1-hour incubation. Incubation of transduced cells occurred as described under “General Cell Culture” above. Following transduction, viral supernatant was aspirated entirely and 2×DPBS wash was performed. Cells were recovered in 5 mL of BHK-21 Full Medium and incubated for 18 to 24 hours.

Virus-containing supernatants were harvested via centrifugation at 500 g for 5 minutes to pellet cellular debris, followed by filtration using a 0.45 μm filter (Merck Millipore #SLHV033RS) and a sterile 10 mL Luer lock syringe (MicroAnalytix, #MS S3P10LL). Clarified supernatants were collected for titration and used for subsequent transduction experiments or stored at 4° C. for up to 1 month.

### Transgene Isolation

In total, 500 μl of viral supernatant was isolated with the MagMAX™ Viral RNA Isolation Kit (ThermoFisher, #AM1939) as per the manufacturer's recommendations. Transgene-specific primers (SEQ ID NO. 20 and 21, 32 and 33, or 42 and 43) anchored to the 5′ and 3′ end of the gene of interest GFP were used for gene-specific RT-PCR using 300 ng of input viral RNA and the SuperScript™ IV One-Step RT-PCR System (ThermoFisher, #12594025). Specifically, reverse transcription was carried out under the following parameters: 50° C. for 1 min, 51° C. for 1 min, 52° C. for 1 min, 53° C. for 1 min, 54° C. for 1 min, 55° C. for 3 mins, 56° C. for 3 mins, 57° C. for 3 mins, 58° C. for 3 mins, 59° C. for 3 mins, 60° C. for 10 mins, 50° C. for 30 mins, and denaturation at 98° C. for 2 mins. Cycling PCR was performed using the following parameters: 98° C. for 10 secs, 50° C. for 10 secs, 72° C. for 30 s per 1 kb for 40 cycles, and 72° C. for 5 mins.

### SINV Mutation Rate Sequencing

SINV-EGFP-ENVELOPE VIRAL RNA (SEQ ID NO. 29), SIN2A-BSR-Envelope (SEQ ID NO. 40), or SIN2A-BSR (C56X)-Envelope (SEQ ID NO. 41) were serially passaged for up to 5 rounds by applying 1 mL of undiluted, clarified virus to transfected cells. Briefly, naïve BHK-21 cells were seeded in T25 flasks at 2.5×10⁵ cells/flask and incubated for 24 hours. Next, BHK-21 cells were transduced with 1 mL of undiluted, clarified viral particles (SEQ ID NO. 29, 40, or 41) and washed as described in “Viral transduction for serial passaging” above. Immediately, cells were transfected with 2.5 μg each of in vitro-transcribed pSinCapsid IncRNA (SEQ ID NO. 27) using 20 μL of Lipofectamine™ 2000 (ThermoFisher #11668019) in 500 μL of Opti-MEM (ThermoFisher #31985070) 35 as per the manufacturer's recommendations. After 4 hours, cells were washed twice with DPBS and switched to 5 mL BHK-21 Full Medium. Transduction and recovery were performed as per “Viral transduction for serial passaging” above, with a recovery volume of 5 mL.

Viral RNA was isolated as described in the “Transgene Isolation section”. For all subsequent steps, DNA concentration was determined using the Qubit™ 1× dsDNA HS Assay Kit (ThermoFisher, #Q32851) following purification or PCR-related steps. 300 ng of viral RNA was amplified using the SuperScript™ IV One-Step RT-PCR System. Bands of interest were gel extracted with the Monarch DNA Gel Extraction Kit (NEB, #T1020). For all subsequent PCRs, ultra-high-fidelity Platinum™ SuperFi II PCR Master Mix (ThermoFisher, #12368050) was used following the manufacturer's instructions. Next, a limited 2-cycle PCR was performed on 100 ng of DNA template (SEQ ID NO. 30) using modified dual-UMI primers (SEQ ID NO. 22 and 23) to append 36 nt UMIs with a branched NNNYR motif. Alternatively, a single 50 nt UMI with a branched NNNYR motif was appended onto an isolated transgene using a single forward, nested primer (SEQ ID NO. 44) on a transgene that was previously amplified via qPCR using gene-specific primers (SEQ ID NO. 42 and 43). Illumina-specific adapters (Illumina, #FC-131-2003) were attached using 15-cycles of PCR following 1.0× Ampure XP bead clean-up prior to sequencing using a NovaSeq 6000 S4 SP PE250 flow cell (Illumina, #20039236).

### Processing of Deep Sequenced Samples

Deep sequencing analysis was performed using the UMIC-seq pipeline. Dual UMIs (SEQ ID NO. 22 and 23) were extracted using both the forward and reverse primer EGFP probe sequence (SEQ ID NO. 20 and 21) and were merged using the matchumi.py script for full clustering (script available upon request). Clustered reads were filtered to enrich for fragments between 400 and 450 bp (-m 400 -M 450) using seqkit for uniform coverage, and filtered reads were mapped using the default parameters of minimap2. Variants were called using LoFreq at a minimum allele frequency of 0.0025. Concomitantly, a plasmid sample of EGFP (SEQ ID NO. 30) was also subjected to deep sequencing under the same PCR parameters as described above. To account for PCR errors, identified variants that overlapped in the plasmid sample of EGFP for the same position and type of nucleotide substitution were excluded from sequencing data. Mutation frequency was determined by dividing the sum of the allele frequency for each mutation by the length of the transgene amplicon (379 bp). Mutation frequency is reported as median with lower and upper range.

### Serial Passaging of SP6 RNA Polymerase to Alter Promoter Specificity Profile

Viral RNA encoding the SIN2A-SP6-Envelope construct (SEQ ID NO. 31) was prepared as described under “Packaging of SINV Particles (referred to as Round 0 (R0))—mRNA Electroporation” and “Viral transduction for serial passaging.” Specifically, however, the SIN2A-SP6-Envelope construct was linearized and in vitro transcribed as RNA, purified and then nucleofected into naïve 1×10⁶ BHK-21 cells using a 1:1 ratio of pSinCapsid (SEQ ID NO. 27) and SIN2A-SP6-Envelope (SEQ ID NO. 46). Subsequently, serial passage of the SIN2A-SP6-Envelope occurred across seven consecutive rounds. For the first two rounds, genetic drift was allowed to occur by serially passaging the viral particles on the SP6 DNA complement (SEQ ID NO. 37), wherein an SP6 promoter drives the expression of pSinCapsid (SEQ ID NO. 27). Between round 3 and round 5, a 1:1 ratio of transfected DNA plasmids including the SP6 DNA complement and a T7 DNA complement (SEQ ID NO. 38) wherein a T7 promoter drives the expression of pSinCapsid was used to transfect BHK-21 cells as described under “Viral transduction for serial passaging.” Between round 6 to round 7, only the T7 DNA compliment (SEQ ID NO. 38) was used prior to viral transduction.

### Quantification of Viral Titre Using E2-Specific Probes

Titration and quantification of viral particles were performed using TaqMan Fast Virus 1-Step Master Mix (Thermo Fisher, #4444434) as outlined in English, J. G. et al. Cell 178, 748-761.e17 (2019). Specifically, 10 μL of clarified, viral supernatant was used with an E2-specific primer probe pair (SEQ ID NO. 24, 25, and 26) that was mutually exclusive to any gene-sequence occurring in the complement viral components (SEQ ID NO. 37 and 38) and quantified on QuantStudio™ 3 Real-Time PCR System (Thermo Fisher, #A28567).

### Preparation of Nanopore Sequencing Libraries

For all subsequent steps, DNA concentration was determined using the Qubit™ 1× dsDNA HS Assay Kit (ThermoFisher, #Q32851) following purification. Ultra-high-fidelity Platinum™ SuperFi II PCR Master Mix (ThermoFisher, #12368050) was used following the manufacturer's instructions for PCR-related steps. Briefly, following RT-PCR as described in Transgene isolation, the amplicon of the gene of interest was gel extracted from a 1% TAE (1× Tris-acetate-EDTA) agarose gel. Next, a limited 2-cycle PCR was performed using 100 ng of input DNA template (SEQ ID NO. 34) using a modified forward UMI primer (SEQ ID NO. 35) to append a 50 nt UMI with a branched NNNYR motif to the amplicon (SEQ ID NO. 34). These modified primers (SEQ ID NO. 36 and 45) also include Gibson overhangs homologous to a destination site in a pUC19 vector, allowing for immediate downstream Gibson assembly following PCR. Gibson assembly was performed using 50 ng of input, PCR-amplified pUC19 vector template (SEQ ID NO. 39) using NEBuilder® HiFi DNA Assembly Master Mix (NEB, #E2621L), and a 3:1 ratio of insert: vector amount for assembly. Transformation into competent cells was performed following manufacturer's protocol (NEB® Stable Competent E. coli, NEB, #C3040H). Subsequent steps are followed as previously described by Zurek and colleagues (Nat. Commun. 11, 6023 (2020)).

Following harvest of the transformed bacterial colonies and restriction digestion to excise the UMI-tagged amplicon (SEQ ID NO. 34), custom barcodes were added onto the amplicon via a limited 2-cycle PCR. Nanopore library preparation was performed as followed for the Ligation Sequencing Kit (ONT, #SQK-LSK109). Briefly, the upper limit of 300 fmol of input amplicon DNA was used for DNA repair and end-prepare using NEBNext FFPE DNA Repair Mix (NEB, #M6630L), wherein the reaction volume was doubled to 120 μL. For the reaction, 7 μL of NEBNext FFPE DNA Repair Buffer (NEB, #M6630L), 4 μL NEBNext FFPE DNA Repair Mix (NEB, #M6630L), 7 uL Ultra II End-prep reaction buffer (NEB, #E7546L), 6 μL Ultra II End-prep enzyme mix (NEB, #E7546L), 1 μL DNA CS (ONT, #SQK-LSK109) and H₂O was added. The reaction was incubated on a thermocycler at 20° C. for 20 mins followed by 65° C. for 20 mins as previous described in Sevim, V. et al. Scientific Data 6, 1-9 (2019).

Subsequent clean-up steps involving 1.0× AMPure XP magnetic beads (Beckman Coulter, #A63881) leveraged extended 30 mins to 1 hour incubation times. Next, end-prepared DNA samples (SEQ ID NO. X) were ligated with nanopore-specific adapters using NEBNext Quick T4 DNA ligase (NEB, #M0202S). The reaction was performed as follows: 60 μL DNA sample, 25 μL Ligation Buffer (LNB) (ONT, #SQK-LSK109), 10 μL NEBNext Quick T4 DNA Ligase (NEB, #E6056L), 10 μL Adapter Mix (ONT, #SQK-LSK109). The reaction volume was incubated at 25° C. for 20 minutes, followed by 4° C. overnight and subsequent clean-up using the Short Fragment Buffer (SFB) (ONT, #SQK-LSK109) and a 1:1 ratio of Ampure XP bead to reaction volume ratio. A final elution volume of 15 μL was used prior to loading onto the nanopore at 40 fmol with the following specifications for the reaction volume: 37.5 μL Sequencing Buffer (SQB) (ONT, #SQK-LSK109), 25.5 μL Loading Beads (LB) (ONT, #SQK-LSK109) and 12 μL of DNA library. The R9.4.1 flow cell was primed using a priming mix including 30 μL of Flush Tether (ONT, #SQK-LSK109) and a vial of Flush Buffer (FB) (ONT, #SQK-LSK109). Subsequently, 800 μL of this priming mix was loaded into the priming port followed by a 5 mins incubation before the library was added into the sample port.

### Base Calling Nanopore Data

Nanopore libraries were run on R9.4 flow cells (FLO-MIN106) under default parameters using ONT MinION. The following input was specified: Voltage, −190 mV; kit used, SQK-LSK109; 1 hour between MUX scans; base calling, enabled; high-accuracy basecalling. Base calling was done using MinIT (ONT-MinIT Release 19.06.08). Alignment and processing of nanopore output was performed with NanoPlot, NanoFilt (minimum q-score set to 10.0), minimap2 and the UMIC-seq protocol as previously described in Zurek, P. J., et al. Nat. Commun. 11, 6023 (2020).

### Plaque Assay for the Determination of Plaque-Forming Units Per mL

Plaque assays were performed in 12-well plates with viral supernatant stocks diluted to concentrations between 10⁻¹ to 10⁻⁶. Briefly, pre-sterilised methylcellulose (Spectrum, #ME136) was dissolved in BHK-21 Full Medium (1% w/v) at 4° C. overnight with agitation (300 RPM) using a magnetic stirrer (referred to henceforth as Plaquing Media). One day prior to viral transduction, naïve BHK-21 cells were plated at a density of 70,000 cells/well in a 12-well plate and incubated as described in “General Cell Culture” above. Diluted or undiluted viral aliquots (200 μL applied to cells) were used to transduce naïve, untransfected BHK-21 cells as described in “Viral transduction and serial passaging” above. Next, 1 mL of 37° C. pre-warmed Plaquing Media was applied to the transduced cell monolayer and the plate was returned to the incubator as described in “General Cell Culture” above and incubated for up to 48 hours without disturbance. After two days, the Plaquing Media was aspirated and the cell monolayer was washed once with 1× DPBS. The cell monolayer was then fixed with 10% neutral buffered formalin for 30 minutes before staining with 0.1% crystal violet (% w/v; Sigma Aldrich, #C0775-25G).

### Machine-Learning Approach Towards the Quantification of Images for PFU and GFP/Brightfield Determination

Briefly, ilastik, a machine-learning approach towards bioimage analysis, was used for the purposes of determining the plaque-forming unit (PFU) and cell density counts using the Cell Density Counting module as previously described (Berg, S. et al. Nat. Methods 16, 1226-1232 (2019)). The following ‘Features’ were selected from the Cell Density Counting interface: for Color/Intesity: 0.30, 0,70. 1.00, 1.60, 3.50, 5.00 and 10.00 for σ0, σ1, σ2, σ3, σ4, σ5, σ6, respectively; for Edge: 0.70, 1.60, and 5.00 for σ1, σ3, and σ5, respectively; and for Texture: 1.00, 3.50, and 10.00 for σ2, σ4, and σ6, respectively. Under the ‘Counting’ interface, a RandomForest algorithm was used (Ntrees: 10, and MaxDepth: 50), with a sigma value of 2.50.

For the determination of PFU, images were firstly cropped to show only a single well of a given dilution and saved in.png format. Using several images in which there were clearly defined plaques in the 10⁻³ or 10⁻⁵ dilution, plaques were indicated as objects in the ‘Foreground’, whereas the contours of the well, as well as areas in which crystal-violet stained cell density was dense, were blocked out as the ‘Background.’ For images in which plaques were more ubiquitous, the well contours were blocked out as described above; however, areas that contained large plaques were indicated entirely as the ‘Foreground.’ For images in which no plaques were evident, the algorithm was trained to recognize that the entire well was the ‘Background’, and this process was iteratively done until the total density field was a value less than 3 in conditions in which no plaques were evident.

For the determination of the GFP+/Brightfield image ratio, ilastik was trained as described above for PFU; however, separate modules were used for GFP+ images and brightfield images. For GFP+ images, images were firstly acquired using a Leica DMIRB (Inverted Leica Modulation Contrast Microscope, Leica) with 5× objective lens, DAPI filter, 2 seconds exposure time, and a field-of-view (FOV) with limited impingement from edges of the T25 flask. The same FOV was used for the corresponding brightfield image, with auto-exposure settings used for image capture. Briefly, raw images (.jpeg format) were uploaded to ilastik, and GFP+ cells were indicated in the ‘Foreground’, whereas the ‘Background’ was indicated by areas devoid of a GFP+ signal. For cell density counting for bright field images, individual foci were indicated as ‘Foreground’ in areas in which cell surface area was greatest, whereas ‘Background’ was indicated using a fine ‘1’ setting ‘size’ for the intervening spaces of cells in images that had adequately sparse density.

For all images, density was exported as a.csv format, and sorted via a custom Rscript for the generation of heatmaps using the ComplexHeatmap package in R.

### Method for Producing a Recombinant Viral Particle

A gene of interest is inserted into a viral vector that is capable of being used as a template to express viral RNA. The gene of interest is inserted into the viral vector using common, scarless molecular cloning techniques that can include Gibson cloning, golden gate cloning. Following molecular manipulation to insert the gene-of-interest, the recombinant viral vector is transformed into competent bacterial host cells. Nucleic acid is purified from the transformed bacterial host cells and the recombinant viral vector is sequenced to identify insertion of the gene of interest. The recombinant viral vector is linearized and used as a template for in vitro transcription of viral RNA that can be used to produce a recombinant viral vector.

A vector bearing a bacterial origin of replication including a derivative of pMB1, pBR322, ColE1, p15A, pSC101, R6K, or F1 as well as an antibiotic selectable marker under a bacterial promoter, as well as a promoter sequence pertaining to either SP6, T7, or T3 sequences for in vitro transcription of a recombinant viral polynucleotide related to alphaviral cDNA sequence.

The alphaviral cDNA sequence encodes the 5′UTR portion, nonstructural genes 1 to 4 (nsp1-4), a partial capsid fragment of up to 225 nucleotides in-frame, a self-cleaving protein signal, a gene of interest, a self-cleaving 2A protein sequence, followed by the full-length polynucleotide sequence of E3, E2, 6K, E1, hereafter referred to as the viral glycoproteins, followed by a 3′UTR portion, a polyadenosine nucleotide tract of 17-35 nucleotides and a restriction digestion site for linearization of the plasmid.

The alphaviral cDNA sequence encodes the 5′UTR portion, nonstructural genes 1 to 4 (nsp1-4), the full-length, in-frame coding sequence of the capsid gene, a self-cleaving 2A protein sequence, gene of interest, followed by the in-frame truncation of the viral glycoproteins, this time including the first 123 nucleotides of E1, followed by the in-frame fusion of the last 129 nucleotides of the E1 gene, followed by a 3′UTR portion, a polyadenosine nucleotide tract of 17-35 nucleotides and a restriction digestion site for linearization of the plasmid. Scarless cloning such as Gibson assembly or gene-synthesis methods are used to insert a gene of interest into a region upstream of viral glycoproteins in place of the viral capsid protein portion of the recombinant viral poynucleotide in an initial construct. Specifically, the insertion of the gene of interest occurs at a region downstream of the 26SP portion of nonstructural protein 4 (nsp4) of an alphaviral polynucleotide cDNA sequence, and further downstream of the short translational ramp including the first 225 nucleotides of an alphaviral capsid protein. The gene of interest is inserted strictly in-frame to the open reading frame (ORF) of the viral capsid protein fragment and glycoprotein components. Specifically, following the first 225 nucleotides of the capsid gene fragment, a self-cleaving protein fragment (such as, but not limited to, the 2A protease signal from foot-and-mouth-disease virus) is inserted, followed by the ATG starting methionine codon of the gene of interest. Strictly, the stop codon of the gene of interest is not included in the ORF, which is instead replaced by a self-cleaving protein fragment, followed by the in-frame sequence of viral glycoproteins. Performing quality-control check on the extracted intact plasmid via standard sequencing procedures that include Sanger sequencing at a region upstream or downstream or within the gene of interest to ensure that it has been inserted as an intact ORF

Following synthesis or cloning of this initial polynucleotide construct, the construct is transformed into electrocompetent or chemically competent bacteria for downstream screening. This method involves picking transformed bacterial colonies plated on selective media relevant to the vectors as described above and processing these samples via extraction of purified plasmid DNA via miniprep, midiprep, maxiprep, or megaprep.

The purified DNA polynucleotide is then linearized using a restriction endonuclease specific to the restriction site occurring downstream of the polyadenosine nucleotide tract as described above; linearized DNA is then prepared via phenol-chloroform extraction.

### A Method for Producing a Virus

Recombinant viral RNA is combined in a 1:1 ratio with purified RNA (or complement) derived from a defective helper plasmid. The defective helper plasmid provides the missing component (in this case, the capsid gene) that has been removed from the recombinant viral RNA polynucleotide. The two RNA species are introduced via electroporation or lipofection into a suitable host cell. The host cells are incubated under general cell culture conditions for a period of up to 24 hours. During this time, infectious viral particles are produced, and this is excreted into the cell culture media which is available for harvesting. The cell culture media is collected and purified of contaminants such as cell debris.

Linearized, purified plasmid DNA bearing polynucleotide sequences as described above is used as input reaction in an in vitro transcribed reaction to produce long non-coding RNA (IncRNA) that is polyadenylated and 5′ capped. This polynucleotide sequence is the engineered, non-naturally occurring (as a result of synthetic manipulation via the aforementioned cloning procedures) alphaviral polynucleotide particle. The resulting in vitro transcribed RNA is LiCI purified and quantified via spectrophotometry.

Simultaneously, a complement polynucleotide bearing the sequence that was replaced by the gene of interest (either the coding sequence of capsid or viral glycoproteins) is also in vitro transcribed to produce a linear, long, non-coding RNA species that is to be co-introduced with the engineered, non-naturally occurring alphaviral polynucleotide sequence. The resulting in vitro transcribed RNA is LiCI purified and quantified via spectrophotometry.

Using a process of electroporation or nucleofection, both species of IncRNA including the engineered, non-naturally occurring alphaviral polynucleotide sequence and the complement component is co-introduced into an amenable host cell for a period of 24 hours, followed by subsequent clarification of the viral supernatant via centrifugation and filtration using a 0.45 μm filter.

### A Method for Selection Stringency during Viral-Assisted Evolution of a Gene of Interest

Naïve, untransduced cells are transfected with a mammalian expression vector that encodes a missing component from the viral particle. After 6 hours to 1 day, the viral particles are applied to the cell. The virus interacts with the mammalian expression vector, which encodes a condition that allows for the expression of the missing viral component. Depending on how easy it is for that condition to be addressed, the virus is able to complete its lifecycle, mature and leave the cell. The virus generates mutations in the gene of interest as it does this, and only those variants of the viral particles that meet the condition of the mammalian expression vector are able to exit the cell. The virus can be clarified and collected from the cell culture and purified to isolate the gene of interest.

A polynucleotide sequence including either the expression of the ORF of the capsid protein or the viral glycoproteins is constitutively or inducibly expressed from a vector that is co-transfected with accessory plasmids, hereafter referred to as ‘stringent circuit’ condition.

A polynucleotide sequence including either the expression of a IncRNA including a truncated or full-length, in-frame fusion of non-structural gene 1 and non-structural gene 4, followed by the full-length or partial sequence of capsid or the viral glycoproteins is constitutively or inducibly expressed from a vector that is co-transfected with accessory plasmids, hereafter referred to as ‘relaxed circuit’ condition.

Both ‘stringent’ and ‘relaxed’ conditions are mutually exclusively transfected into an amenable host cell in separate T25 flasks, which are then co-selected and treated with the engineered viral particle simultaneously and monitored for growth or decay of the population via qPCR-based methods of quantification, plaque assays, or using RNA biosensors.

### A Method of Performing Circuitous Evolution of a Gene of Interest

The introduction of a mammalian expression plasmid that encodes for the conditional expression of the missing component of the virus is first introduced into an amenable host cell. The viral particles containing the gene of interest are added to these amenable host cells. The gene of interest contained within the recombinant viral particle interacts with the mammalian expression plasmid. Some embodiments of the gene of interest develop mutations as a result of being in the viral particle/viral vector. These mutations confer a benefit to these gene of interest variants in terms of unlocking the conditional expression for the missing viral component. Expression of the missing viral component thereby confers an advantage to the viral particle encoding that particular variant of the gene of interest. Evolved viral particles containing mutated gene of interest are able to enter the media. Following collection of cell media, the process can be repeated iteratively, or the gene-of-interest can be isolated and sequenced.

Transfection of ‘stringent’ and ‘relaxed’ circuits conditions is performed on an amenable host cell in mutually exclusive T25 flasks. After a period of 6 hours to 1 day following this transfection event, media is aspirated and viral supernatant containing engineered, non-naturally occurring viral particles are introduced onto the cell at a multiplicity of infection (MOI) of 1 for transduction, or by applying 1 mL of undiluted viral supernatant.

The T25 flask is incubated at 37° C. for one hour, with intermittent shaking every 15 minutes to allow even coverage of the surface area of the cell monolayer.

Subsequently, the viral supernatant is aspirated, and 2× DPBS wash is performed to remove residual viral supernatant. Culture media is replenished and the T25 flask are returned to the incubator for a period of up to 1 to 2 days.

Media is harvested, hereafter referred to as ‘viral supernatant’, and clarified as described above. The viral supernatant is stored at refrigerated conditions.

Viral supernatant is again applied to cells that have been transfected as described above is repeated.

### Insertion of a Blasticidin Resistance Gene

Insertion of a blasticidin resistance gene containing a stop codon at position 56 (C56X) upstream from viral structural genes results in a 110-fold increase in the allele frequency of the wildtype revertant, and a further 39-fold increase in the allele frequency of a mutation encoding the most commonly used codon trinucleotide in BHK-21 host cells encoding for cysteine. In yet other embodiments, the insertion of the gene of interest in-frame with the partial or full coding sequence of the downstream structural genes of the non-competent viral vector enables the codon optimization of the isolated gene of interest (e.g., optimized for expression in mammalian cells) by increasing the frequency of optimal codons (i.e., optimal for the host cell) in synonymous mutations by at least 2 orders of magnitude in the gene of interest that is to be evolved by circuitous evolution.

### Interpretation

In addition, where features or aspects of the invention are described in terms of Markush groups, those skilled in the art will recognise that the invention is also thereby described in terms of any individual member or subgroup of members of the Markush group.

For the purpose of this specification, where method steps are described in sequence, the sequence does not necessarily mean that the steps are to be carried out in chronological order in that sequence, unless there is no other logical manner of interpreting the sequence.

Unless specifically stated otherwise, it is appreciated that throughout the specification discussions utilizing terms such as “processing”, “computing”, “calculating”, “determining”, “analysing” or the like, refer to the action and/or processes of a computer or computing system, or similar electronic computing device, that manipulate and/or transform data represented as physical, such as electronic, quantities into other data similarly represented as physical quantities.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to one of ordinary skill in the art from this disclosure, in one or more embodiments.

Similarly, it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Descriptions, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

As used herein, unless otherwise specified the use of the ordinal adjectives “first”, “second”, “third”, etc., to describe a common object, merely indicate that different instances of like objects are being referred to and are not intended to imply that the objects so described must be in a given sequence, either temporally, spatially, in ranking, or in any other manner.

In the description provided herein, numerous specific details are set forth. However, it is understood that embodiments of the invention may be practiced without these specific details. In other instances, well-known methods, structures, and techniques have not been shown in detail in order not to obscure an understanding of this description.

In describing the preferred embodiment of the invention illustrated in the drawings, specific terminology will be resorted to for the sake of clarity. However, the invention is not intended to be limited to the specific terms so selected, and it is to be understood that each specific term includes all technical equivalents which operate in a similar manner to accomplish a similar technical purpose. Terms such as “forward”, “rearward”, “radially”, “peripherally”, “upwardly”, “downwardly”, and the like are used as words of convenience to provide reference points and are not to be construed as limiting terms.

For the purposes of this specification, the term “plastic” shall be construed to mean a general term for a wide range of synthetic or semisynthetic polymerization products, and generally consisting of a hydrocarbon-based polymer.

As used herein the term “and/or” means “and” or “or”, or both.

As used herein “(s)” following a noun means the plural and/or singular forms of the noun.

In the claims which follow and in the preceding description of the invention, except where the context requires otherwise due to express language or necessary implication, the word “comprise” or variations such as “comprises” or “including” are used in an inclusive sense, i.e., to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means including.

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

It is apparent from the above, that the arrangements described are applicable to at least the biotechnology and medical research industries.

1. A nucleic acid sequence selected from the group consisting of:
a first nucleic acid sequence that comprises 5′ to 3′:
at least part of a 5′ UTR sequence,
at least part of one or more non-structural protein(s) coding sequence,
at least part of a sub-genomic promoter coding sequence,
at least part of a capsid coding sequence,
at least part of a first protease cleavage signal coding sequence,
a gene of interest coding sequence,
at least part of a second protease cleavage signal coding sequence,
at least part of an E3 coding sequence,
at least part of an E2 coding sequence,
at least part of a 6K coding sequence,
at least part of an E1 coding sequence, and
at least part of a 3′ UTR sequence,
wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus;
a second nucleic acid sequence that comprises 5′ to 3′:
at least part of a 5′ UTR sequence,
at least part of one or more non-structural protein(s) coding sequence,
at least part of a sub-genomic promoter coding sequence,
at least part of a capsid protein coding sequence,
at least part of one or more structural protein(s) coding sequence(s), and
at least part of a 3′ UTR sequence,
wherein the 5′ UTR sequence, the one or more non-structural protein(s) coding sequence, the capsid protein coding sequence, the one or more structural protein(s) coding sequence(s), and the 3′ UTR sequence are coding sequences of a Togaviridae virus; and
a third nucleic acid sequence that comprises 5′ to 3′:
at least part of a capsid protein coding sequence,
at least part of one or more structural genes coding sequence(s), and
at least part of a 3′ UTR sequence,
wherein the capsid protein coding sequence, the one or more structural proteins coding sequence(s), and the 3′ UTR sequence are sequences of a Togaviridae virus.

2. The nucleic acid sequence according to claim 1, wherein the Togaviridae virus is an Alphavirus.

3. The nucleic acid according to claim 1, wherein the gene of interest coding sequence and the sub-genomic promoter coding sequence are operably linked.

4. The nucleic acid according to claim 1, wherein each of the first protease cleavage signal coding sequence and the second protease cleavage signal is a self-cleaving peptide coding sequence.

5. The nucleic acid according to claim 1, wherein each of the first protease cleavage signal coding sequence and the second protease cleavage signal is independently selected from the group consisting of equine rhinitis A virus E2A coding sequence, foot-and-mouth disease virus F2A coding sequence, porcine teschovirus-1 2A P2A coding sequence, and those assigned as virus 2A T2A coding sequence.

6. The nucleic acid according to claim 1, wherein the one or more structural protein(s) coding sequence(s) is at least a part of the E3 structural protein coding sequence.

7. The nucleic acid according to claim 1, wherein the one or more structural protein(s) coding sequence(s) is at least a part of the E2 structural protein gene coding sequence.

8. The nucleic acid according to claim 1, wherein the one or more structural protein(s) coding sequence(s) is at least a part of the 6K structural gene coding sequence.

9. The nucleic acid according to claim 1, wherein the one or more structural protein(s) coding sequence(s) is at least a part of the E1 structural gene coding sequence.

10. The nucleic acid according to claim 1, wherein the gene of interest encodes a biomolecule.

11. The nucleic acid according to claim 1, wherein the gene of interest encodes at least one precursor of a biomolecule.

12. A method of evolution of a gene product of a gene of interest, the method comprising:
providing one or more viral vector(s) comprising 5′ to 3′ at least part of a 5′ UTR sequence, at least part of one or more non-structural protein(s) coding sequence, at least part of a sub-genomic promoter coding sequence, at least part of a capsid coding sequence, at least part of a first protease cleavage signal coding sequence, a gene of interest coding sequence, at least part of a second protease cleavage signal coding sequence, at least part of an E3 coding sequence, at least part of an E2 coding sequence, at least part of a 6K coding sequence, at least part of an E1 coding sequence, and at least part of a 3′ UTR sequence, wherein the 5′ UTR sequence, the one or more non-structural protein(s) coding sequence, the capsid coding sequence, the E3 coding sequence, the E2 coding sequence, the 6K coding sequence, the E1 coding sequence, and the 3′ UTR sequence are sequences of a Togaviridae virus;
providing one or more first complement nucleic acid sequence(s) comprising 5′ to 3′ at least part of a 5′ UTR sequence, at least part of one or more non-structural protein(s) coding sequence, at least part of a sub-genomic promoter coding sequence, at least part of a capsid protein coding sequence, at least part of one or more structural protein(s) coding sequence(s), and at least part of a 3′ UTR sequence into the population of suitable host cells, wherein the 5′ UTR sequence, the one or more non-structural protein(s) coding sequence, the capsid protein coding sequence, the one or more structural genes coding sequence(s), and the 3′ UTR sequence are sequences of a Togaviridae virus, and wherein expression of the one or more first complement nucleic acid sequence(s) is linked to a function of a gene product of the gene of interest;
introducing the one or more viral vector(s) to a population of suitable host cells;
introducing the one or more first complement nucleic acid sequence(s) to the population of suitable host cells;
allowing maturation and egress of one or more mature virus(es) that comprise the gene of interest or a variant thereof from the population of suitable host cells;
recovering the one or more mature virus(es);
introducing the one or more mature virus(es) and one or more first complement nucleic acid sequence(s) into a population of naïve suitable host cells;
allowing further maturation and egress of further one or more mature virus(es) that comprise the gene of interest or a variant thereof from the population of suitable host cells;
recovering the further one or more mature virus(es);
isolating one or more nucleic acid sequence(s) from the further one or more mature virus(es) to provide one or more isolated nucleic acid sequence(s); and
isolating the gene of interest or a variant thereof from the one or more isolated nucleic acid sequence(s).

13. The method of evolution of a gene product of a gene of interest according to claim 12, wherein the Togaviridae virus structural protein coding sequence is selected from the group consisting of the capsid coding sequence, the E3 coding sequence, the E2 coding sequence, the 6K coding sequence, and the E1 coding sequence of a Togaviridae virus.

14. The method of evolution of a gene product of a gene of interest according to claim 12, wherein the one or more structural protein(s) coding sequence(s) is at least a part of the E3 structural protein coding sequence.

15. The method of evolution of a gene product of a gene of interest according to claim 12, wherein the one or more structural protein(s) coding sequence(s) is at least a part of the E2 structural protein gene coding sequence.

16. The method of evolution of a gene product of a gene of interest according to claim 12, wherein the one or more structural protein(s) coding sequence(s) is at least a part of the 6K structural gene coding sequence.

17. The method of evolution of a gene product of a gene of interest according to claim 12, wherein the one or more structural protein(s) coding sequence(s) is at least a part of the E1 structural gene coding sequence.
